(19) United States
(12) Patent Application Publication
(10) Pub. No.: US 2006/0083726 A1
(43) Pub. Date: Apr. 20, 2006

(54) Method for controlling insects of the order diptera using a Bacillus thuringiensis strain

(75) Inventors: Timothy James Lysyk, Lethbridge (CA); Leonard Brent Selinger, Lethbridge (CA); Lisa Dawn Kalischuk-Tymensen, Lethbridge (CA); Richard Charles Lancaster, Lethbridge (CA); Danica Darlene Sonja Baines, Lethbridge (CA)

Correspondence Address: GREENLEE WINNER AND SULLIVAN P C, 4875 PEARL EAST CIRCLE, SUITE 200, BOULDER, CO, 80301, US

(73) Assignee: Her Majesty the Queen in Right of Canada as Rep. by the Minister of Agriculture & Agri-Food, Lethbridge (CA)

(21) Appl. No.: 11/254,249

(22) Filed: Oct. 19, 2005

Related U.S. Application Data
(60) Provisional application No. 60/620,019, filed on Oct. 19, 2004.
(60) Provisional application No. 60/675,132, filed on Apr. 27, 2005.

(51) Int. Cl. A01N 63/00; C12N 1/20

(52) U.S. Cl. 424/93.461; 435/252.31

(57) ABSTRACT

The invention provides a method for controlling insects of the Order Diptera by providing a Bacillus thuringiensis strain or variant thereof, or a spore or crystal of the Bacillus thuringiensis strain or variant thereof, and either contacting the insect with or administering to an animal the Bacillus thuringiensis strain or variant thereof, or the spore or crystal of the Bacillus thuringiensis strain or variant thereof; or applying the Bacillus thuringiensis strain or variant thereof, or the spore or crystal of Bacillus thuringiensis strain or variant thereof to an infested area. The Bacillus thuringiensis strain contains a plasmid carrying endotoxin genes for encoding delta-endotoxins Cry1A, Cry1B, Cry1F, Cry1H, Cry1I, Cry1K, Cry2 or a variant thereof. Preferably, the strain is Bacillus thuringiensis strain LRC3 deposited as ATCC PTA-6248. Methods for preparing the strain, spores, crystals, mutants, variants, and compositions incorporating same are described.

### CROSS REFERENCE TO RELATED APPLICATIONS

This application claims priority from U.S. Provisional Patent Application No. 60/620,019 filed Oct. 19, 2004 and U.S. Provisional Patent Application No. 60/675,132 filed Apr. 27, 2005. To the extent that it is consistent herewith, the aforementioned applications are incorporated herein by reference.

### FIELD OF THE INVENTION

The invention pertains to a method for controlling insects of the Order Diptera using a strain of Bacillus thuringiensis which produces insecticidal crystals effective against such insects. Specifically, the invention relates to methods for preparing and using the strain, spores, crystals and variants thereof, and compositions incorporating same.

### BACKGROUND OF THE INVENTION

Bacillus thuringiensis (Bt) is a Gram-positive, facultative, spore-forming, and rod-shaped bacterium which produces insecticidal crystals during sporulation. These crystals generally contain from three to seven proteins referred to as delta-endotoxins (known commercially as “Bt toxins”) in inactive or protoxin forms, the combination of which dictates insect specificity. Unlike conventional chemical insecticides which generally kill through non-specific contact with a target insect, Bt-based products must be ingested by insects with a generally alkaline (reducing environment) midgut (pH range of 10-12) and specific gut membrane structures required to bind the delta-endotoxin. Not only must the insects have the correct physiology and be at a susceptible stage of development, but also the bacterium must be consumed in sufficient quantity.

Bt-based products require a specific set of interactions with a target insect to cause death. The insect must initially ingest the crystals which then travel to the midgut. Upon entering the midgut, the crystals are solubilized as a result of a high reducing capacity of the digestive fluid (pH 10). The released protoxins are then cleaved by a gut protease to produce active toxins termed delta-endotoxins. The delta-endotoxins interact with digestive cells lining the midgut, causing leakage of the cells. Such leakage disrupts general insect homeostasis mechanisms, ultimately causing insect death.

Bt strains produce two types of toxin, namely the Cry (crystal) toxins encoded by different cry genes, and the Cyt (cytolytic) toxins which can augment the activity of Cry toxins, enhancing the effectiveness of insect control. Several successful Bt varieties or Bt-based products are presently commercially available for controlling immature stages of Lepidoptera (Bt kurstaki, Bt aizawa), aquatic Diptera (Bt israeliensis), and Coleoptera (Bt tenebrionis). Over forty classes of Bt toxins have been identified, but only six classes are present in current commercial formulations:
Bt kurstaki-Cry1Aa, Cry1Ab, Cry1Ac, Cry1Ia, Cry2A
Bt aizawa-Cry1Ab, Cry1Ac, Cry1C,
Bt israeliensis-Cry4A, Cry4B, Cry11, CytA; and
Bt tenebrionis-Cry3.

While most of the Bt strains produce delta-endotoxins which share a common basic toxin structure, they differ in insect host range, perhaps due to different degrees of binding affinity to the toxin receptors in the insect gut; for example, the toxins produced by Bt aizawai are somewhat different from those of Bt kurstaki and their host range differs, but are highly specific to insects of the Order Lepidoptera, with no effect on other insects. Very few Bt toxins have been assessed in greater detail as to their effects on different insect groups.

The Order Diptera is an extensive order of insects having two functional wings, two balancers, and mouthparts modified for sucking or piercing. Such insects undergo a complete metamorphosis with larval, pupal and adult stages. Diptera are divided into three large groups: Nematocera (e.g., black fly, crane fly, gnat, midge, mosquito, and sand fly); Brachycera (e.g., bee fly, deer fly, horse fly and robber fly); and Cyclorrhapha (flies that breed in vegetable or animal material, both living and dead e.g., bottle fly, blow fly, cattle grub, deer ked, face fly, fruit fly, house fly, horn fly, horse louse fly, human bot fly, rodent fly, rabbit bot fly, sheep ked, sheep nasal bot, stable fly, stomach bot and Tsetse).

Animals exposed to such flies exhibit economically significant weight loss resulting from the feeding behaviour and mechanical interaction with adult flies. In general, adult flies are associated with manure and decaying straw near the animals, whereas larvae may be found in different locations. Although several chemical products are available for controlling the activity of adult flies, they are generally ineffective or have adverse effects on the environment. Indoor confined larvae and outdoor confined larvae are found in accumulated manure. As a chemical product for controlling indoor confined larvae, Cyromazine™ or Larvidex™ has been found to be ineffective. There are currently no registered products for control of outdoor confined larvae. Outdoor unconfined larvae are found in isolated manure in pastures. Two chemical products (e.g., Dimilin™ or Ivermectin™) used to control these larvae are effective, but have adverse effects on other arthropods. Additionally, regular use of chemicals to control unwanted insects can select for chemically resistant strains, a problem which has occurred in many species of economically important pests. De-registration of the remaining few chemical insecticides due to adverse effects upon animals and humans makes it imperative to develop new technology.

A further problem with current Bt preparations resides in the narrow host range. Bt strains have been described for use against fire ants (U.S. Pat. No. 6,551,800 to Bulla, Jr. et al.); tobacco hornworm (U.S. Pat. No. 5,308,760 to Brown et al.; Schnepf and Whitley, 1981); nematodes (U.S. Pat. No. 4,948,734 to Edwards et al. and U.S. Pat. No. 5,151,363 to Payne); Coleoptera, specifically the cotton boll weevil, Colorado potato beetle, alfalfa weevil, Egyptian alfalfa weevil (U.S. Pat. Nos. 4,797,276 and 4,853,331 to Herrnstadt et al., U.S. Pat. No. 4,999,192 to Payne et a. and U.S. Pat. No. 4,849,217 to Soares et al.); Lepidoptera, specifically Pieris brassicae (large white butterfly), Spodoptera littoralis (Mediterranean climbing cutworm), Heliothis virescens (tobacco budworm), Memestra brassicae (cabbage moth) (Sanchis et al., 1988; Visser et al., 1990; U.S. Pat. No. 6,448,226 to Lambert et al., U.S. Pat. No. 6,570,005 to Schnepf et al., and U.S. Pat. No. 6,593,293 to Baum et a.). Within the Order Diptera, U.S. Pat. No. 5,888,503 to Hickle et al. describes Bt strains effective only against insects of the family Calliphoridae (screw-worm and sheep blowfly), while U.S. Pat. No. 6,482,636 to Donovan et al. describes a Bt israeliensis strain which is toxic to mosquito larvae. To date, current Bt strains and preparations thereof are generally limited to a few, particular insects within an Order, but not to all members of such an Order.

Although a narrow host range may be advantageous in targeting a specific insect, limited toxicity reduces the use of such products. As multiple insects are found on crops or other infested areas, a broad spectrum insecticide would be both efficient and cost-effective. Additionally, current commercial products eradicate predominantly immature insects, but fail to affect adult insects. An insecticide which accomplishes control of both immature and adult insects is most desirable. Further, a product of biological origin would exhibit significant advantages over chemical insecticides in being easily manufactured, safe, inexpensive and commercially valuable.

### SUMMARY OF THE INVENTION

The present invention provides a method for controlling an insect of the Order Diptera using a strain of Bacillus thuringiensis by contacting the insect with the strain or a variant thereof; a spore or variant thereof from the strain; a crystal or variant thereof from the strain; or a crystal containing delta-endotoxins Cry1A, Cry1B, Cry1F, Cry1H, Cry1I, Cry1K, Cry2 or a variant thereof. Preferably, the strain is Bacillus thuringiensis strain LRC3 deposited as ATCC PTA-6248.

Broadly, the invention thus provides a method for controlling an insect of the Order Diptera comprising the step of:

a) providing a Bacillus thuringiensis strain or a variant thereof, or a spore or a crystal of the Bacillus thuringiensis strain or a variant thereof, the Bacillus thuringiensis strain containing a plasmid carrying one or more endotoxin genes for encoding one or more delta-endotoxins selected from Cry1A, Cry1B, Cry1F, Cry1H, Cry1I, Cry1K, Cry2 or a variant thereof; and one of the following steps selected from:

b) contacting the insect with the Bacillus thuringiensis strain or the variant thereof, or the spore or the crystal of the Bacillus thuringiensis strain or the variant thereof;

c) applying the Bacillus thuringiensis strain or the variant thereof, or the spore or the crystal of Bacillus thuringiensis strain or the variant thereof to an infested area; or

d) administering the Bacillus thuringiensis strain or the variant thereof, or the spore or the crystal of Bacillus thuringiensis strain or the variant thereof to an animal.

In another aspect, there is provided a composition for controlling an insect of the Order Diptera comprising a Bacillus thuringiensis strain or a variant thereof, or a spore or a crystal of Bacillus thuringiensis strain or a variant thereof. The Bacillus thuringiensis strain contains a plasmid carrying one or more endotoxin genes for encoding one or more delta-endotoxins selected from Cry1A, Cry1B, Cry1F, Cry1H, Cry1I, Cry1K, Cry2 or a variant thereof.

In another aspect, there is provided a crystal of a Bacillus thuringiensis strain or a variant thereof for use in controlling an insect of the Order Diptera. The crystal contains one or more delta-endotoxins selected from Cry1A, Cry1B, Cry1F, Cry1H, Cry1I, Cry1K, Cry2 or a variant thereof.

In another aspect, there is provided an isolated nucleic acid of a Bacillus thuringiensis strain or a variant thereof, wherein the nucleic acid encodes a protein toxic to an insect of the Order Diptera. The protein is selected from Cry1A, Cry1B, Cry1F, Cry1H, Cry1I, Cry1K, Cry2 or a variant thereof.

In another aspect, there is provided a plasmid of the Bacillus thuringiensis strain or a variant thereof, comprising one or more endotoxin genes selected from cry1A, cry1Bb, cry1Fb, cry1Hb, cry1Ic, cry1ka, cry2 or a variant thereof. Vectors and host cells comprising the nucleic acid or plasmid are further provided.

In yet another aspect, there is provided a Bacillus thuringiensis strain for controlling insects of the Order Diptera, wherein the strain contains a plasmid carrying one or more endotoxin genes for encoding one or more delta-endotoxins selected from Cry1A, Cry1B, Cry1F, Cry1H, Cry1I, Cry1K, Cry2 or a variant thereof.

As used herein and in the claims, the terms and phrases set out below have the following definitions:

“Animal” is meant to include, for example, dairy and beef cattle, pigs, goats, sheep, horses; deer, buffalo, elk, chickens, turkeys, as well as domestic animals such as cats, dogs, and horses. The term is meant to include young and adult animals.

“Bacillus thuringiensis” abbreviated as “Bt” is meant to refer to a specific Gram-positive bacterium which during sporulation, produces parasporal protein crystals having insecticidal properties.

“Crystal” or “crystals” is meant to refer to protein of the parasporal crystals formed in Bacillus thuringiensis species. The protein can be inactive (protoxin) or active (delta-endotoxin or Bt toxin).

A “delta-endotoxin” or or “endotoxin” or “Bt toxin” means an insecticidal toxin produced by any Bacillus thuringiensis species.

A “DNA fingerprint” of a single Bt strain is obtained from using genomic fingerprinting techniques that exploit the various repetitive DNA sequences found in bacterial genomes. These techniques use single oligonucleotide primers targeting repetitive sequences that are interspersed in the genome to create variable-sized amplified DNA fragments. The fragment profile on an agarose gel is consequently used to fingerprint the bacteria of interest down to the species/strain level. The three amplification techniques used herein are REP (Repetitive Extragenic Palindromic), ERIC (Enterobacterial Repetitive Intergenic Consensus) and RAPD (Random Amplified Polymorphic DNA).

Two polynucleotides or polypeptides are “homologous” or “identical” if the sequence of nucleotides or amino acid residues, respectively, in the two sequences is the same when aligned for maximum correspondence as described herein. Sequence comparisons between two or more polynucleotides or polypeptides are generally performed by comparing portions of the two sequences over a comparison window to identify and compare local regions of sequence similarity. The comparison window is generally from about 20 to about 200 contiguous nucleotides or contiguous amino acid residues. The “percentage of sequence identity” or “percentage of sequence homology” for polynucleotides and polypeptides may be determined by comparing two optimally aligned sequences over a comparison window, wherein the portion of the polynucleotide or polypeptide sequence in the comparison window may include additions or deletions (i.e., gaps) as compared to the reference sequence (which does not comprise additions or deletions) for optimal alignment of the two sequences. The percentage is calculated by: (a) determining the number of positions at which the identical nucleic acid base or amino acid residue occurs in both sequences to yield the number of matched positions; (b) dividing the number of matched positions by the total number of positions in the window of comparison; and, (c) multiplying the result by 100 to yield the percentage of sequence identity.

Optimal alignment of sequences for comparison may be conducted by computerized implementations of known algorithms, or by inspection. A list providing sources of both commercially available and free software is found in Ausubel et al. (2000). Readily available sequence comparison and multiple sequence alignment algorithms are, respectively, the Basic Local Alignment Search Tool (BLAST) and ClustalW programs. For greater certainty, as used herein and in the claims, “percentage of sequence identity” or “percentage of sequence homology” of amino acid sequences is determined based on optimal sequence alignments determined in accordance with the default values of the BLASTP program, available as described above.

As discussed in greater detail hereinafter, homology between nucleotide sequences can also be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology.

“Host cell” includes an animal, a plant, a yeast, a fungal, a protozoan and a prokaryotic host cell.

“Infested area” means an area affected by insects of the Order Diptera. The term is meant to include accumulated manure, manure patties, decomposing material, manure pits, sewage lagoons, bedding debris, leaves, crops or other environments where the immature and adult insects are associated.

“Insecticidally effective amount” means an amount of the Bt strain or variant thereof, or spore or crystal of the strain or a variant thereof which is capable of controlling or eradicating an insect of the Order Diptera as measured by percent mortality, absence of further crop damage, or absence of further weight reduction in an animal.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

“LD₅₀” means “lethal dose” and is the amount of a material administered all at once which causes the death of 50% of a group of test animals. The LD₅₀ is a method to measure the short-term poisoning potential or acute toxicity of a material.

A “microarray” gene assessment is conducted by hybridizing DNA from a Bt strain to a microarray system. For example, the test used herein (developed at the Biotechnology Research Institute, Montreal, Quebec, Canada) detects the cry1A genes down to the tertiary rank as well as providing primary rank information on cry1, cry2, cry3, cry4, cry9 and cry11 genes.

A “mutant” or “variant” of a gene refers to nucleotide sequences which encode for the same Bt toxins or which encode for functionally equivalent Bt toxins which are capable of controlling or eradicating an insect of the Order Diptera.

A “mutant” or “variant” of a protein means variants (including derivatives or analogs) having the same or essentially the same biological activity against an insect of the Order Diptera as the exemplified delta-endotoxins. Such variants may differ in amino acid sequence from the delta-endotoxin by one or more substitutions, additions, deletions, fusions, and truncations, which may be present in any combination, without altering the capacity of the variants to control or eradicate insects of the Order Diptera.

A “mutant” or “variant” of a strain means variants of the Bt strain having the same or essentially the same characteristics or biological activity against an insect of the Order Diptera as the exemplified Bt strain.

“Order Diptera” or “Diptera” is meant to include Diptera of the following groups: Nematocera (e.g., black fly, crane fly, gnat, midge, mosquito, and sand fly); Brachycera (e.g., bee fly, deer fly, horse fly and robber fly); and Cyclorrhapha (flies that breed in vegetable or animal material, both living and dead e.g., bottle fly, blow fly, cattle grub, deer ked, face fly, fruit fly, house fly, horn fly, horse louse fly, human bot fly, rodent fly, rabbit bot fly, sheep ked, sheep nasal bot, stable fly, stomach bot and Tsetse).

A “polynucleotide” or “nucleic acid” means a linear sequence of deoxyribonucleotides (in DNA) or ribonucleotides (in RNA) in which the 3′ carbon of the pentose sugar of one nucleotide is linked to the 5′ carbon of the pentose sugar of the adjacent nucleotide via a phosphate group. The “polypeptide” or “nucleic acid” may comprise DNA, including cDNA, genomic DNA, and synthetic DNA, or RNA, which may be double-stranded or single-stranded, and if single-stranded, may be the coding strand or non-coding (anti-sense) strand.

A “polypeptide” or “protein” means a linear polymer of amino acids that are linked by peptide bonds.

A “plasmid” means an extrachromosomal covalently continuous double-stranded DNA molecule which occurs in bacteria.

A “protoxin” means a precursor protein which must be solubilized in the midgut and enzymatically activated to be effective.

A “spore” means a reproductive body, often a single cell, which is capable of development into an adult organism.

“Toxin” means a solubilized, enzymatically processed protein which can cause insect death.

“Toxic” means the ability to control or eradicate immature and adult insects of the Order Diptera.

“Transformation” means the directed modification of the genome of a cell by the external application of purified recombinant DNA from another cell of different genotype, leading to its uptake and integration into the subject cell's genome. In bacteria, the recombinant DNA is not integrated into the bacterial chromosome, but instead replicates autonomously as a plasmid.

A “transgenic” means an organism into which foreign DNA has been introduced into the germ line.

A “transgenic plant” encompasses all descendants, hybrids, and crosses thereof, whether reproduced sexually or asexually, and which continue to harbour the foreign DNA, and is meant to include transgenic plants, plant tissues, and plant cells.

A “vector” means a nucleic acid molecule that is able to replicate autonomously in a host cell and can accept foreign DNA. A vector carries its own origin of replication, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include, but are not limited to, phage, cosmid, baculovirus, retroviral, and plasmid vectors.

### BRIEF DESCRIPTION OF THE DRAWINGS

FIG. 1 shows the protein banding patterns for crystals isolated from Bt strain LRC3 (lane 2), Bt kurstaki (HD1-lane 3) and Bt israeliensis (4Q5-lane 4), with molecular weight standards (kDa) in lane 1 (myosin 191, phosphorylase 97, bovine serum albumin 64, glutamic dehydrogenase 51, alcohol dehydrogenase 39, carbonic anhydrase 28, myoglobin red 19, lysozyme 14).

FIG. 2 shows the DNA fingerprint of Bt strain LRC3 compared to several type strains of Bacillus thuringiensis using REP-1R and 21 primers. Eight μl of each reaction was loaded per lane. Lane M, DNA size ladder in kilobases (in kb). Lane 1, B. thuringiensis subsp. kurstaki 4D1; lane 2, B. thuringiensis subsp. israeliensis HD-500; lane 4, Bt strain LRC3 (Agriculture and Agri-Food Canada); lane C, control (-DNA template). A=REP-1R primer; B=REP-21 primer; C=REP-1R and REP-2I primers.

FIG. 3 shows the DNA fingerprint of Bt strain LRC3 compared to several type strains of Bacillus thuringiensis using Bc-REP-1 and Bc-REP-2 primers. Eight μl of each reaction was loaded per lane. Lane M, DNA size ladder (in kb). Lane 1, B. thuringiensis subsp. kurstaki 4D1; lane 2, B. thuringiensis subsp. israeliensis HD-500; lane 4, Bt strain LRC3 (Agriculture and Agri-Food Canada); lane C, control (-DNA template). A=Bc-REP-1 primer; B=Bc-REP-2 primer; C=Bc-REP-1 and Bc-REP-2 primers.

FIG. 4 shows the DNA fingerprint of Bt strain LRC3 compared to several type strains of Bacillus thuringiensis using ERIC-1R and ERIC-2 primers. Eight μl of each reaction was loaded per lane. Lane M, DNA size ladder (in kb). Lane 1, B. thuringiensis subsp. kurstaki 4D1; lane 2, B. thuringiensis subsp. israeliensis HD-500; lane 4, Bt strain LRC3 (Agriculture and Agri-Food Canada); lane C, control (-DNA template). A=ERIC-2 primer; B=ERIC-1R primer; C=ERIC-2 and ERIC-1R primers.

FIG. 5 shows the DNA fingerprint of Bt strain LRC3 compared to several type strains of Bacillus thuringiensis using BOX-A1R and ERIC-2 primers. Eight μl of each reaction was loaded per lane. Lane M, DNA size ladder (in kb). Lane 1, B. thuringiensis subsp. kurstaki 4D1; lane 2, B. thuringiensis subsp. israeliensis HD-500; lane 4, Bt strain LRC3 (Agriculture and Agri-Food Canada); lane C, control (-DNA template). A=BOX-A1R primer; B=BOX-A1R and ERIC-2 primers.

FIG. 6 shows the DNA fingerprint of LRC3 compared to several type strains of Bacillus thuringiensis using RAPD primers (0955-03,1940-12,1910-08). Eight μl of each reaction was loaded per lane. Lane M, DNA size ladder (in kb). Lane 1, B. thuringiensis subsp. kurstaki 4D1; lane 2, B. thuringiensis subsp. israeliensis HD-500; lane 4, Bt strain LRC3 (Agriculture and Agri-Food Canada); lane C, control (-DNA template). A=0955-03 primer; B=1940-12 primer; C=1910-08 primer.

FIG. 7 shows the microarray key for assessing the gene content of Bt strain LRC3, Bacillus thuringiensis subsp. kurstaki, and Bacillus thuringiensis subsp. israeliensis.

FIG. 8 shows the microarray assay results for Bacillus thuringiensis subsp. kurstaki HD-1.

FIG. 9 shows the microarray assay results for Bacillus thuringiensis subsp israeliensis HD-500.

FIG. 10 shows the microarray assay results for Bt strain LRC3.

### DETAILED DESCRIPTION OF THE INVENTION

This invention broadly relates to a method for controlling an insect of the Order Diptera by providing a Bacillus thuringiensis strain or a variant thereof, or a spore or crystal of the Bacillus thuringiensis strain or a variant thereof, and either contacting the insect with or administering to an animal, the Bacillus thuringiensis strain or the variant thereof, or the spore or crystal of the Bacillus thuringiensis strain or the variant thereof; or applying the Bacillus thuringiensis strain or the variant thereof, or the spore or crystal of Bacillus thuringiensis strain or the variant thereof to an infested area. Preferably, the strain is Bacillus thuringiensis strain LRC3 deposited as ATCC PTA-6248. It will be appreciated that Bacillus thuringiensis strains having similar characteristics to Bacillus thuringiensis strain LRC3 as described herein; plasmids, isolated nucleic acids, proteins, mutants or variants thereof; and compositions are within the scope of the present invention.

The Bacillus thuringiensis strain of the present invention was originally obtained from the Bacillus Genetic Stock Center (Department of Biochemistry, Ohio State University, 484 West Twelve Avenue, Colombus, Ohio, U.S.A. 43210). It is a wild-type strain and was deposited Oct. 6, 2004 in the American Type Culture Collection under Accession Number PTA-6248. Characteristics of Bt strain LRC3 are set forth in Table 1.

TABLE 1
Characteristics of the Bt LRC3 strain compared to Bt israeliensis

| | Inclusion | |
|---|---|---|
| Strain | Type | H-antigen |
| Bt israeliensis | amorphic | 14 |
| Bt strain LRC3 | pyramidal | 12 |

Bt strain LRC3 is a Gram-positive, facultative, spore-forming, and rod-shaped bacterium, with a distinctive feature being one or more protein inclusions which form adjacent to the spore. These inclusions appear microscopically as distinctively shaped crystals, which assist in differentiating Bt strains; for example, Bt strain LRC3 produces pyramidal crystals, while Bt israeliensis produces amorphic crystals. Bt strains can be further classified immunologically on the basis of cell surface antigens such as the H-antigen (flagellar antigen).

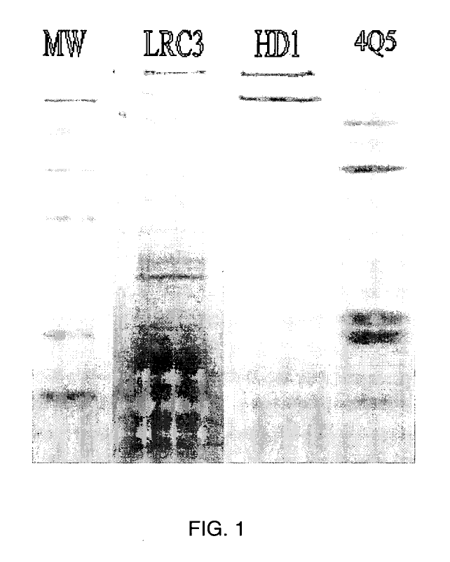

In addition, Bt strains can be distinguished from their unique Bt toxin protein profiles. Bt strains produce two types of toxin, namely the Cry (crystal) toxins encoded by different cry genes and the Cyt (cytolytic) toxins which can augment the activity of Cry toxins. The crystals are composed of proteins which are inactive (protoxin) and can be roughly distinguished by their molecular weights on a standard SDS-PAGE gel; for example, when such proteins are separated on SDS-polyacrylamide gels, major bands generally appear in the molecular weight range of 120-140 kDa, with additional groups of bands in the 60-70 kDa and 23-30 kDa ranges. A protein profile for Bt strain LRC3 is shown in FIG. 1 and described in Example 2. FIG. 1 shows the protein banding patterns for crystals isolated from Bt strain LRC3 (lane 2). Analysis of protein profiles revealed different electrophoretic patterns for each strain. Bt kurstaki HD-1 has about a 191, 120 kDa bands representing Cry1A proteins and a 64 kDa band representing Cry2a protein. Bt israeliensis 4Q5 has bands of about 100 kDa representing Cry4 proteins, a 97 kDa band representing the Cry4B protein, a 64 kDa band representing Cry10 and Cry11 proteins, and a 27-30 kDa bands representing Cyt1 protein. Bt strain LRC3 has about a 120-191 kDa bands representing Cry1A, Cry1B, Cry1F, Cry1K proteins, an 80 kDa protein representing Cry1I protein, a 70 kDa band representing a Cry2 protein, a 55 kDa band representing an undefined protein, 38-48 kDa bands representing an undefined protein, and 25-28 bands representing a Cyt protein.

DNA fingerprinting was performed to distinguish Bt strain LRC3 from the other major Bt strains currently used in Bt formulations. Various hybridization-based, amplification-based or sequence-targeted genomic fingerprinting techniques have been developed to identify single Bt subspecies. Several endogenous, interspersed repetitive DNA elements are conserved in many bacteria. Families of repetitive sequences have been identified, including the repetitive extragenic palindromic (REP) sequence, the enterobacterial repetitive intergenic consensus (ERIC) sequence, and the BOX element (Versalovic et al. 1991a, 1991b). Primers have been designed which enable the amplification of distinct DNA sequences lying between these elements in the polymerase chain reaction (PCR). The PCR fragments are then separated by gel electrophoresis to generate DNA fingerprints which are specific for particular bacteria and enable comparative analyses. Random amplified polymorphic DNA (RAPD) is an alternative method which involves amplifying undefined regions of template DNA using PCR with arbitrary primers. Such methods are useful for discriminating and comparing bacteria. Three different typing methods were used to generate fingerprinting patterns, namely REP, ERIC and RAPD, with each method having its own specialized primer sequences. While DNA fingerprinting methods generally use a single primer to amplify the repetitive sequence, utilization of two primers can yield a third pattern that is not the sum of the two individual primers. This third pattern can elicit a distinction between Bt strains that the single primers do not.

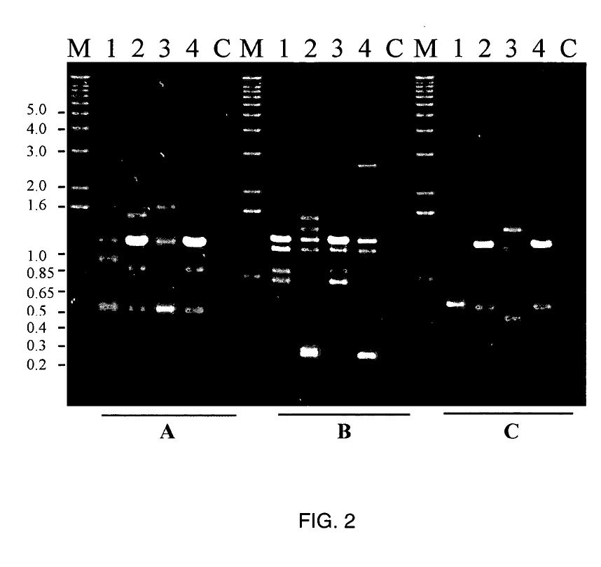

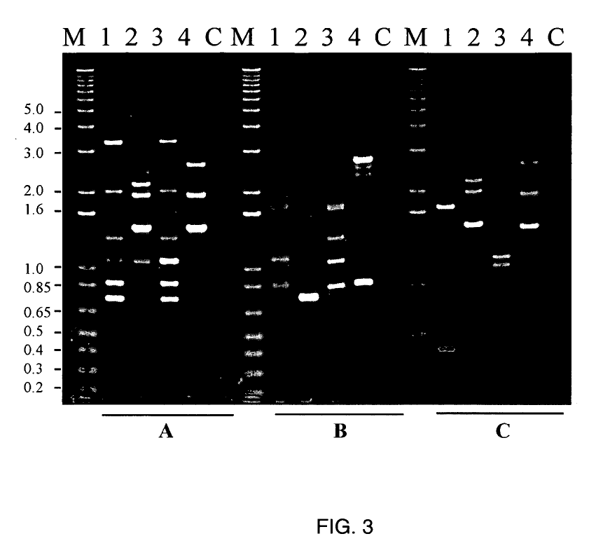

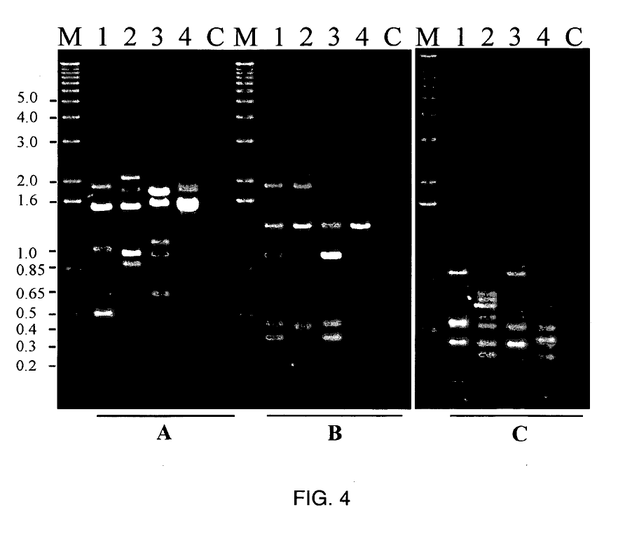

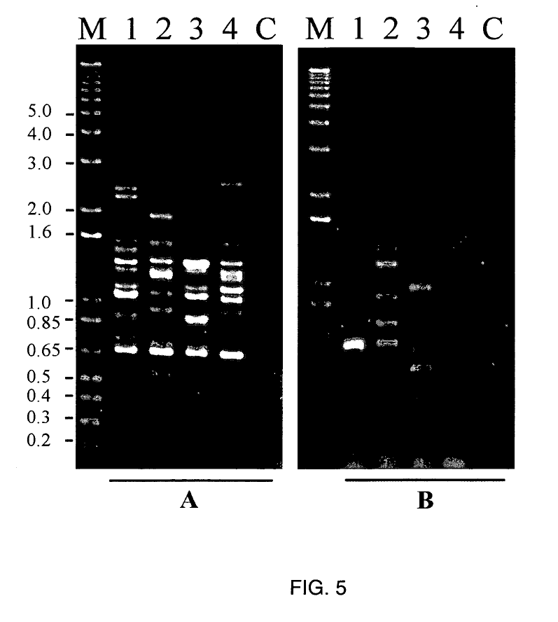

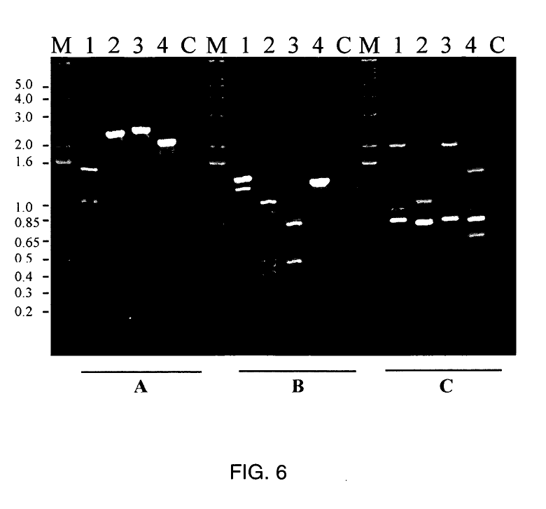

FIGS. 2-6 show the DNA fingerprints of Bt strain LRC3 compared to those of a Bt kurstaki and Bt israeliensis using various techniques and primers (REP amplification using REP-1R and REP-2I primers in FIG. 2; Bc-REP-1 and Bc-REP-2 primers in FIG. 3; ERIC amplification using ERIC-2 and ERIC-1R primers in FIG. 4; BOX-A1R primer in FIG. 5; and RAPD amplification using 0955-03, 1940-12 and 1910-08 primers in FIG. 6). Example 3 discusses the results in further detail. In general, the DNA fingerprint for Bt strain LRC3 can be distinguished from the DNA fingerprint of Bt kurstaki and Bt israeliensis using all three methods.

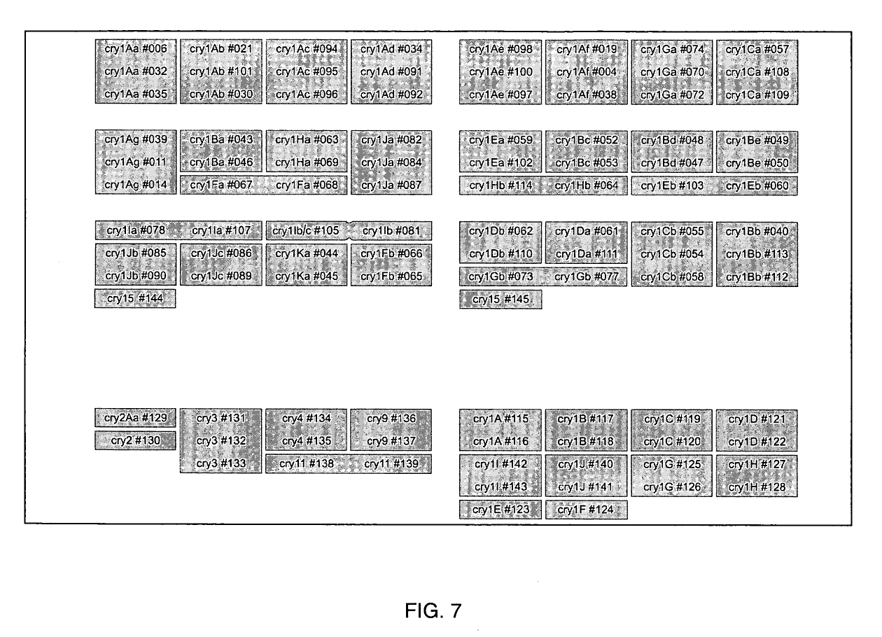

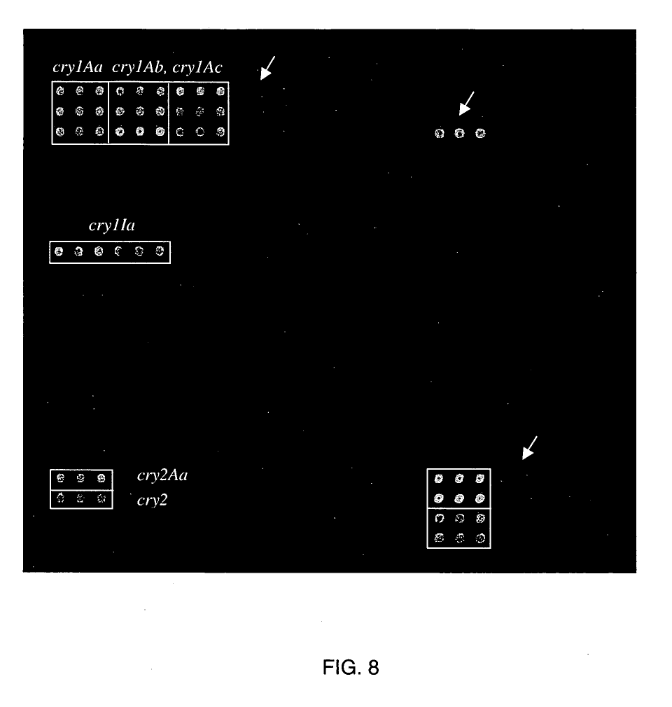

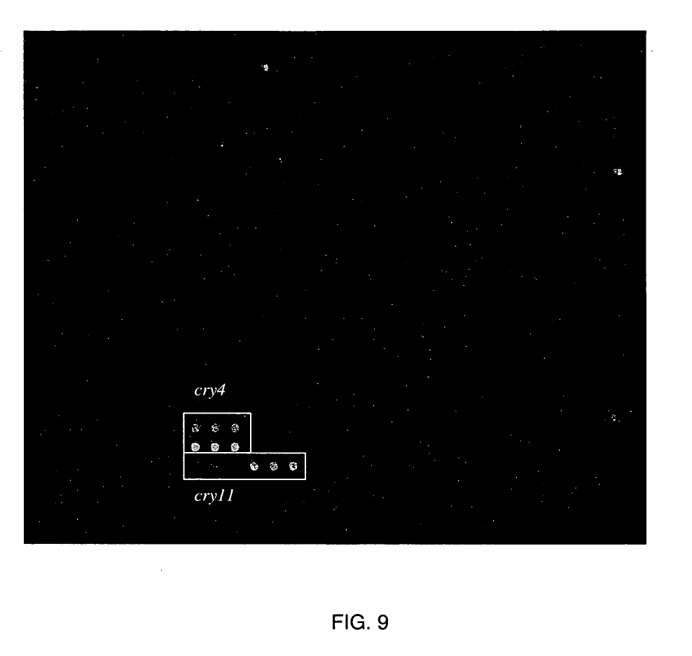

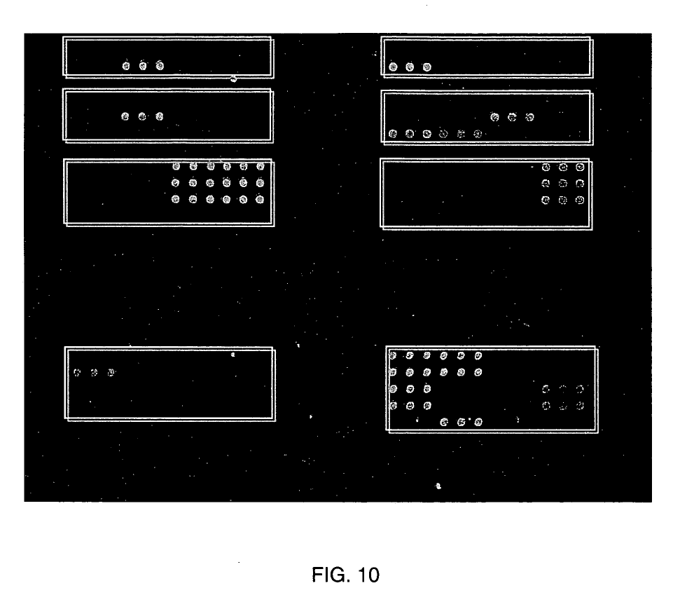

Bt strains produce Cry (crystal) toxins encoded by different cry genes. The cry gene content of Bt strain LRC3 was compared to the cry gene content of the two main types of Bt strains present in commercial formulations, Bt kurstaki and Bt israeliensis. FIG. 7 shows the gene microarray key. FIGS. 8-10 show the gene microarray results for Bt kurstaki, Bt israeliensis and Bt strain LRC3. For clarity, spots corresponding to the presence of particular Cry genes are boxed in accordance with the gene microarray key of FIG. 7. Example 4 discusses the results in further detail. The genes identified in Bt kurstaki are as expected and include the Cry1A genes, Cry1Ia gene and the Cry2Aa gene (FIG. 8). Bt israeliensis also has the expected genes and include the Cry4 and Cry11 genes (FIG. 9). The Cry genes identified for Bt strain LRC3 are Cry1A, Cry1Abb, Cry1Fb, Cry1Hb, Cry1Ic, Cry1Ka, and Cry2 (FIG. 10, Tables 5 and 6 in Example 4).

The ability of Bt strain LRC3 to eradicate insects of the Order Diptera was determined and compared with the insecticidal activity of Bt israeliensis. A problem with current commercial products, notably Bt israeliensis, resides in the narrow host range. Although a narrow host range may be advantageous in targeting a specific insect, limited toxicity reduces the use of such products. Multiple insects are associated with animals, on crops or other infested areas. It will be appreciated that a broad spectrum insecticide will eradicate multiple insects at one time, providing a cost-effective and efficient alternative. Expansion of the host range of an insecticide is thus most desirable. Bt israeliensis has a very narrow host range affecting only immature stages of mosquitoes and blackflies.

However, in comparison to Bt israeliensis, Bt strain LRC3 exhibits a broader spectrum of activity against insects of the Order Diptera which includes Nematocera (e.g., black fly, crane fly, gnat, midge, mosquito, and sand fly), Brachycera (e.g., bee fly, deer fly, horse fly and robber fly) and Cyclorrhapha (flies that breed in vegetable or animal material, both living and dead e.g., bottle fly, blow fly, cattle grub, deer ked, face fly, fruit fly, house fly, horn fly, horse louse fly, human bot fly, rodent or rabbit bot fly, sheep ked, sheep nasal bot, stable fly, stomach bot and Tsetse). Bt strain LRC3 is particularly effective against face flies, fruit flies, horn flies, house flies and stable flies. While Bt israeliensis is effective against only immature insects, Bt strain LRC3 is potent against both adult and immature insects as demonstrated in Examples 5 and 7-9.

The activity of Bt strain LRC3 and crystals thereof were compared to Bt israeliensis and its crystals. The insecticidal activity of Bt strain LRC3 was compared to that of Bt israeliensis by conducting feeding bioassays on adult insects, for example house flies and stable flies (Example 5). Bt strain LRC3 and Bt israeliensis can be used in their entirety (i.e., including spores/crystals/bacterial products). Bt strain LRC3 is active against both adult house and stable flies, whereas Bt israeliensis is ineffective. The effect of purified crystals of Bt strain LRC3 against adult insects (i.e., house flies and stable flies) was tested (Example 7). Based on the LD₅₀ values, adult stable flies appear to be ten times more sensitive to purified crystals of Bt strain LRC3 than are adult house flies. This is expected as each fly species will differ in their sensitivity to the Bt toxins depending on their gut physiology.

Further, the insecticidal activity of Bt strain LRC3 against immature insects (i.e., face flies, fruit flies, horn flies, house flies and stable flies) was tested by conducting rearing and ring bioassays (Example 8). The results demonstrate that both Bt strain LRC3 and Bt israeliensis display similar activity in simple diets. However, Bt strain LRC3 is more effective than Bt israeliensis against non-aquatic immature flies in complex rearing environments, suggesting that Bt strain LRC3 is better able to survive in a complex environment than Bt israeliensis. The insecticidal activity of purified crystals of Bt strain LRC3 against immature insects (i.e., house flies and stable flies) was tested using a ring bioassay (Example 9). Purified crystals of Bt strain LRC3 are more effective than crystals of Bt israeliensis against higher fly larvae. The LD₅₀ for the purified crystals of Bt strain LRC3 was 10 times lower than the LD₅₀ (about 1 ng) which has been reported for Bt israeliensis against mosquito larvae. The differences observed in the activity of the purified crystals of Bt strain LRC3 and the Bt israeliensis strain suggest that the lytic phospholipase C present in the bacterial products may be responsible for most of the activity against house fly larvae (Keller and Langenbruch 1993). The similar effects against fly larvae with the Bt strain LRC3 and delta-endotoxin crystals show that the delta-endotoxin crystals contain the active component responsible for fly larvae death.

Compared to the current commercial Bt israeliensis product, Bt strain LRC3 or spores or crystals thereof are capable of successfully eradicating a broader spectrum of insects at both immature and adult stages. Although the delta-endotoxin (or toxin) protein profiles between Bt strain LRC3 and Bt israeliensis indicate the presence of some similar proteins, Bt strain LRC3 nevertheless has distinct proteins which expand both the range and the age of insects which can be controlled. Hence, an insecticide based on Bt strain LRC3 or spores or crystals thereof would be cost-effective and efficient. As a product of biological origin, Bt strain LRC3 or spores or crystals thereof exhibit significant advantages over chemical insecticides in being nontoxic to predatory insects, birds and mammals, and being easily manufactured using standard fermentation techniques well known in the art.

Bt strain LRC3 can be cultured using standard known media and fermentation techniques. In general, Bt strains grow well on media containing sugars, organic acids, alcohols, or other carbon sources; a nitrogen source such as ammonium; and vitamins if required. The Bt strain can be cultured on many complex media for example, Difco™ nutrient agar, LB agar, TB broth or any other general bacterial growth media. As Bt strain LRC3 grows best aerobically, the cultures are aerated by vigorous shaking the cultures in flasks. In one embodiment, Bt strain LRC3 is used to inoculate fermentation medium comprising bacto-peptone, glucose, potassium phosphate dibasic, potassium phosphate monobasic, and salt solutions (one comprising calcium chloride, manganese chloride, and iron sulphate; and the other comprising magnesium sulphate). The salt solutions are filter sterilized and added to the autoclaved broth at the time of inoculation with the Bt strain LRC3. The flasks are incubated at 28° C. on a rotary shaker at 200 rpm for three days, or until the bacteria produce crystals as verified using a compound microscope at 100× magnification.

Suitable fermentation techniques can be easily scaled-up to industrial fermentors using techniques known in the art. In general, the first step is made using a laboratory fermentor of 5 to 10 litres to test variations in medium, temperature and pH, followed by use of increasingly larger fermentors (e.g., 150,000 gal) for pilot plant and commercial stages.

Preservation of Bt strain cultures is recommended to prevent loss of the plasmids, hence possible loss of crystal production or other plasmid-borne phenotypes, during routine subculturing. For long-term storage of cultures of Bt strains, the pellet obtained from a 3-5 day culture is mixed with glycerol in a 85:15% ratio. Working cultures are stored at −20° C. and long-term storage cultures are stored at −80° C. For long-term storage of the crystals of Bt strains, sodium azide is added to the crystal suspension in water to prevent contamination by microorganisms and the solution is stored at 4° C.

Following fermentation, Bt strains or spores or crystals thereof can be used in various formulations and compositions. Fermentation medium containing the Bt strain, crystals and spores can be readily lyophilized and used in its entirety, while the crystals can be separated from the spores and fermentation medium using procedures known in the art, such as simple centrifugation. Example 6 describes a method for isolating and purifying the crystals from Bt strain LRC3. Further, the step of separating crystals from spores can be omitted by producing asporogenous mutants or variants of the Bt strain using techniques known to those skilled in the art, for example, ultraviolet irradiation or nitrosoguanidine.

The Bt strain, spores or crystals thereof can be formulated as a solid, liquid, suspension, feed additive, admixture, or feed composition as follows:

i) Solids

The Bt strain, spores or crystals thereof can be formulated as a solid, granule, pill, pellet, paste, puck, powder or dust. In the form of a powder or dust, the Bt strain, spores or crystals thereof may be dusted or sprinkled onto accumulated manure, manure patties or decomposing material; manure pits, sewage lagoons, or bedding debris; onto leaves, crops or other environments where the insects are associated; or into feed bunks or mixed with a ration for animals.

ii) Liquids and Suspensions

The Bt strain, spores or crystals thereof can be incorporated into liquids, formulated as solutions or suspensions, by adding lyophilized or powdered Bt strain, spores or crystals to a suitable liquid. Foams, gels, suspensions, emulsions or the like are also suitable. In such forms, the Bt strain, spores or crystals thereof can be sprayed or drenched onto accumulated manure, manure patties or decomposing material; manure pits, sewage lagoons, or bedding debris; or onto leaves, crops or other environments where the insects are associated; or into feed bunks or rations for animals. A solution of the Bt strain, spores or crystals thereof can be applied to susceptible animals by plunge dipping, shower dipping, jetting or spraying. Plunge dipping and shower dipping are effective in saturating the animals; thus, application of a solution of the Bt strain, spores or crystals thereof may not need to be as frequent. However, repeated applications using jetting or spraying may be required to ensure effective wetting.

iii) Feed Additive or Feed Composition

The Bt strain, spores or crystals thereof can be administered indirectly to the environment of the insects in the form of a feed additive or bolus, comprising lyophilized Bt strain, spores or crystals thereof, for young and adult animals including, but not limited to, dairy and beef cattle, pigs, goats, sheep, horses, deer, buffalo, elk, chickens, turkeys, as well as domestic animals such as cats, dogs, and horses. The feed additive may be included with the animals' regular feed material or supplied as a pill or puck.

Incorporation of active ingredients into feed material is commonly achieved by preparing a premix of the active ingredient, mixing the premix with vitamins and minerals, and then adding the premix or feed additive to the feed. The Bt strain, spores or crystals thereof can be admixed with other active ingredients known to those in the art. The active ingredients, including the Bt strain, spores or crystals thereof alone or in combination with other active ingredients, can be combined with nutrients to provide a premixed supplement. The premix may then be added to feed materials. Further, the Bt strain, spores or crystals thereof can be provided in the form of a feed composition comprising a feed material treated with the Bt strain, spores or crystals thereof. The Bt strain, spores or crystals thereof may be mixed with a feed material in dry form; e.g. as a powder, or as a liquid to be used as a drench or spray for example.

The above formulations may incorporate adhesion agents, binders, botanical materials, carriers, detergents, diluents, dispersants, emulsifiers, excipients, extenders, fillers, inorganic minerals, insecticidal carriers, pesticidal additives, polymers, Theological agents, spreader sticker adjuvants, stabilizing agents, surfactants, wetting agents, or combinations thereof. Components which assist in storage stability, handling or administration of the formulation are suitable. Further, it will be appreciated that the Bt strain, spores or crystals thereof can be combined with other cells, crystals, crystal proteins, protoxins, toxins and other insecticides, such as biocides, fertilizers, fungicides and herbicides to provide additional benefits.

The concentration of the Bt strain, spores or crystals thereof will vary on a number of factors depending upon the chosen formulation, method of application, environmental conditions, extent of infestation, or growth stage of the insects. Overall, an effective insecticidal amount is desirable, namely an amount of the Bt strain, spores or crystals thereof which is capable of controlling or eradicating the insects as measured by percent mortality, absence of further crop damage, or absence of further weight reduction in an animal. Specifically, dry formulations may contain from 1-95% by weight or volume of active Bt strain, spores or crystals thereof, more preferably from 20-80% by weight or volume of active Bt strain, spores or crystals thereof, and most preferably 30-60% by weight or volume of active Bt strain, spores or crystals thereof. Liquid formulations may contain 1-60% by weight or volume of active Bt strain, spores or crystals thereof, more preferably from 10-50% by weight or volume of active Bt strain, spores or crystals thereof, and most preferably 20-40% by weight or volume of active Bt strain, spores or crystals thereof. The formulations may contain from 10²-10⁴ spores/ml, more preferably 200-800 spores/ml, and most preferably 300-700 spores/ml. For application to a large area of land, formulations may be administered from about 50 g (dry or liquid) to 1 kg or more per hectare, more preferably from about 50 g (dry or liquid) to 1 kg or more per hectare, and most preferably from about 50 g (dry or liquid) to 1 kg or more per hectare. In field conditions, frequent application might be required to avoid removal of the active material by environmental elements such as wind or precipitation.

The above formulations may be applied to an infested area or susceptible animals in a variety of ways to control or eradicate the insects. The term “infested area” is meant to refer to an area affected by insects of the Order Diptera. In the form of a powder or dust, the Bt strain, spores or crystals thereof may be dusted or sprinkled onto accumulated manure, manure patties or decomposing material; manure pits, sewage lagoons, or bedding debris; onto leaves, crops or other environments where the insects are associated; or into feed bunks or mixed with a ration for animals.

The Bt strain, spores or crystals thereof can also be presented to the insects in a “bait bin,” namely a covered bin containing an attractant, such as blood or fermented milk, which attracts the insects to a food source such that they ingest an efficacious dose of the Bt strain, spores or crystals thereof. Such bins are placed in areas where the insects normally frequent.

Further, pills or pucks of the Bt strain, spores or crystals thereof can be dropped into aquatic environments to release crystals slowly into the environment of the insects. For instance, such pills or pucks can be placed into standing water where the insects, for example mosquitoes, may breed or hatch. Such pills or pucks can also be applied directly to accumulated manure, manure patties or decomposing material; manure pits, sewage lagoons, or bedding debris; onto leaves, crops or other environments where the insects are associated; or into feed bunks or mixed with a ration for animals.

The Bt strain, spores or crystals thereof can be administered indirectly to the environment of the insects in the form of a feed additive or feed composition for animals. The Bt strain, spores or crystals thereof can be provided in the form of a feed additive or feed composition comprising a feed material treated with the Bt strain, spores or crystals thereof. The Bt strain, spores or crystals thereof may be mixed with a feed material in dry form; e.g. as a powder, or as a liquid to coat the feed material. When pills or pucks of the Bt strain, spores or crystals thereof are consumed by animals, the Bt strain, spores or crystals thereof can be indirectly deposited through the animals' manure to accumulated manure, manure patties, or manure pits.

Further, the Bt strain, spores or crystals thereof can be used to control or eradicate insects associated with environments, such as greenhouses, picnic areas, backyards, parks or lakes which are frequented by humans. For example, the Bt strain, spores or crystals thereof can be applied to particular environments to control mosquitoes or blackflies which may be a nuisance to humans. As the Bt strain, spores or crystals thereof is essentially non-toxic to humans, pets and wildlife, it can thus be used in sensitive areas where pesticide use normally causes adverse effects.

The present invention also extends to isolated nucleic acids of a Bacillus thuringiensis strain or a variant thereof, wherein the nucleic acid encodes a protein toxic to an insect of the Order Diptera. The protein is preferably Cry1A, Cry1B, Cry1F, Cry1H, Cry1I, Cry1K, Cry2 or variant thereof. Preferably, the nucleic acid comprises an endotoxin gene selected from cry1A, cry1Bb, cry1Fb, cry1Hb, cry1Ic, cry1Ka, cry2 or a variant thereof. Further, the invention extends to the genes of Bt strain LRC3, including full length sequences and fragments thereof, and use of such genes. Standard techniques known to those skilled in the art can be used to isolate the genes of the Bt strain and the particular genes which encode the crystals (Sambrook et al., 1989; Ausubel et al., 2000). Fragments can be made using commercially available exonucleases or endonucleases according to standard techniques known to those skilled in the art.

Such genes can be introduced into a variety of expression systems. Host cells can include, but are not limited to, an animal, plant, yeast, fungal, protozoan and prokaryotic host cells. Selection of an appropriate host depends upon factors such as gene-host compatibility, expression efficiency, stability and minimal degradation or inactivation of the genes.

Microorganisms which naturally inhabit the growing area of important crops and are known food sources for the insects may be transformed, applied to the growing area and ingested by the insects. Suitable microorganisms which may have faster growing rates than Bt strain LRC3 can be transformed with the gene to expedite production of the desired crystals, or modified to prolong the activity or prevent degradation of the crystals. Further, it might be desirable to transform specific microorganisms which thrive in particular environments affected by the insects; for instance, a microorganism which survives well in water might be effective against larvae of aquatic Dipterans such as mosquitoes. Microorganisms which could be used include, without limitation, Aspergillus niger, Aspergillus ficuum, Aspergillus awamori, Aspergillus oryzae, Bacillus subtilis or licheniformis, Clavibacter xyli, Escherichia coli, Kluyveromyces lactis, Mucor miehei, Pichia pastoris, Pseudomonas fluorescens, Saccharomyces cerevisiae, and Trichoderma reesei.

Insect-resistant transgenic plants can be engineered to express the toxic crystal in their tissues, thereby killing insects which feed on the crops. In regard to animals, expression of the genes in particular plant species provides an economical and direct way to supplement Bt strain LRC3, spores or crystals thereof to susceptible animals. Plant species may include, without limitation, barley, canola, corn, flax, fruit crops, hay grasses, oats, potato, rice, rye, sorghum, tomatoes, vegetables, vine crops, wheat and bedding plants. Preferred plant species for use with the invention are barley, corn, hay grasses and wheat.

A variety of techniques are available for introducing foreign DNA into host cells including, but not limited to, microparticle bombardment, Agrobacterium-mediated transformation, Pseudomonas fluorescens mediated transformation, protoplast transformation, micro-injection, high velocity ballistic penetration and electroporation.

The invention further extends to variants or mutants of the Bt strain, and the genes thereof, including full length sequences and fragments thereof. Preferably, the invention extends to use of variants or mutants of Bt strain LRC3 and genes thereof to control or eradicate insects of the Order Diptera. Mutation of the Bt strain can be induced by techniques known to those skilled in the art including, but not limited to, ultra-violet irradiation or nitrosoguanidine. Cultures can be screened for variants by visual observation of spontaneous mutations such as morphology or color, or identification of the presence of similar characteristics such as the inclusion type, H-antigen, or protein profile. Variants may then be further selected by conventional, small-scale screening for crystal toxicity. Suitable variants may then be optimized for toxin production by using known techniques of yield improvement or manipulating the strain itself to produce mutants, recombinants or genetically engineered derivatives thereof. Such manipulation may also include the preparation of a preferred phenotype of the selected variant; for example, an asporogeneous variant, as obtained through ethylmethane sulfonate mutagenesis, produces crystals but no spores. It will be appreciated that the Bt strain or variants thereof may also be used as starting materials for identifying genetic determinants of a particular toxicity profile, constructing the appropriate gene-specific or sequence-specific probes, and pre-screening other strains at large for the presence of those determinants.

Alternatively, those skilled in the art can alter the genes obtained from the Bt strain, or genes from variants or mutants of the Bt strain through standard mutagenesis techniques, and test altered gene sequences for expression of crystal proteins. Useful mutagenesis techniques known in the art include, without limitation, oligonucleotide-directed mutagenesis, region-specific mutagenesis, linker-scanning mutagenesis, and site-directed mutagenesis by PCR (Sambrook et al., 1989; Ausubel et al., 2000). Homologous nucleotide sequences can be determined by DNA hybridization analysis. Two polynucleotides or polypeptides are “homologous” or “identical” if the nucleotides or amino acid residues, respectively, in the two sequences is the same when aligned for maximum correspondence as described herein. Sequence comparisons between two or more polynucleotides or polypeptides are generally performed by comparing portions of the two sequences over a comparison window to identify and compare local regions of sequence similarity. The comparison window is generally from about 20 to about 200 contiguous nucleotides or contiguous amino acid residues. The “percentage of sequence identity” or “percentage of sequence homology” for polynucleotides and polypeptides may be determined by comparing two optimally aligned sequences over a comparison window, wherein the portion of the polynucleotide or polypeptide sequence in the comparison window may include additions or deletions (ie. gaps) as compared to the reference sequence (which does not comprise additions or deletions) for optimal alignment of the two sequences. The percentage is calculated by: (a) determining the number of positions at which the identical nucleic acid base or amino acid residue occurs in both sequences to yield the number of matched positions; (b) dividing the number of matched positions by the total number of positions in the window of comparison; and, (c) multiplying the result by 100 to yield the percentage of sequence identity.

Optimal alignment of sequences for comparison may be conducted by computerized implementations of known algorithms, or by inspection. “Percentage of sequence identity” or “percentage of sequence homology” of amino acid sequences is determined based on optimal sequence alignments determined in accordance with the default values of the BLASTP program, available as described above. Homology between nucleotide sequences can also be determined by DNA hybridization analysis, wherein the stability of the double-stranded DNA hybrid is dependent on the extent of base pairing that occurs. Conditions of high temperature and/or low salt content reduce the stability of the hybrid, and can be varied to prevent annealing of sequences having less than a selected degree of homology.

Further, the invention extends to a method for controlling or eradicating insects of the Order Diptera using variant or mutant crystals of the Bt strain. Variants may be created using standard known techniques (Sambrook et al., 1989; Ausubel et al., 2000). Those skilled in the art will recognize that proteins may be modified by certain amino acid substitutions, additions, deletions, and post-translational modifications, without loss or reduction of biological activity. In particular, it is well-known that conservative amino acid substitutions, that is, substitution of one amino acid for another amino acid of similar size, charge, polarity and conformation, are unlikely to significantly alter protein function. The 20 standard amino acids that are the constituents of proteins can be broadly categorized into four groups of conservative amino acids as follows: the nonpolar (hydrophobic) group includes alanine, isoleucine, leucine, methionine, phenylalanine, proline, tryptophan and valine; the polar (uncharged, neutral) group includes asparagine, cysteine, glutamine, glycine, serine, threonine and tyrosine; the positively charged (basic) group contains arginine, histidine and lysine; and the negatively charged (acidic) group contains aspartic acid and glutamic acid. Substitution in a protein of one amino acid for another within the same group is unlikely to have an adverse effect on the biological activity of the protein.

It will be apparent to those of ordinary skill in the art that alternative methods, reagents, procedures and techniques other than those specifically detailed herein can be employed or readily adapted to practice this invention. The invention is further illustrated in the following non-limiting Examples. All abbreviations used herein are standard abbreviations used in the art. Specific procedures not described in detail in the Examples are well-known in the art.

### EXAMPLE 1

### Characteristics and Culturing of the Bt Strain LRC3

The Bacillus thuringiensis strain was originally obtained from the Bacillus Genetic Stock Center (Department of Biochemistry, Ohio State University, 484 West Twelve Avenue, Colombus, Ohio, U.S.A. 43210). This wild-type strain was deposited on Oct. 6, 2004 in the American Type Culture Collection under Accession Number PTA-6248. Bt strain LRC3 was used to inoculate the following fermentation medium:

TABLE 2
Composition of medium used for culturing Bt LRC3 strain

| Composition | Concentration | |
|---|---|---|
| Bacto Peptone | 7.5 g/l | |
| Glucose | 1.0 g/l | |
| K₂HPO₄ | 4.35 g/l | |
| KH₂PO₄ | 3.4 g/l | |
| Salt solution #1 | 5 ml/l broth comprising: | 2M CaCl₂ (29.4 g), |
| | | 10⁻²M MnCl (0.223 g), and |
| | | 10⁻³M FeSO₄ (0.093 g) |
| Salt solution #2 | 5 ml/l broth comprising: | 2 M MgSO₄ (49.2 g) |

Salt solutions #1 and #2 were filter sterilized and added to the autoclaved broth at the time of inoculation with the Bt strain LRC3. The flasks were incubated at 28° C. on a rotary shaker at 200 rpm for 3 days, or until the bacteria produced crystal protein as verified under a light microscope at 100× magnification (oil).

### EXAMPLE 2

### Protein Delta-Endotoxin Crystals Purified from Bt Strain LRC3, Bt kurstaki and Bt israeliensis

The Bt kurstaki and Bt israeliensis strains were obtained from the Bacillus Genetic Stock Center under BGSC accession number 4D1 and 4Q5, respectively. Protein profiles using SDS-PAGE were conducted to provide a crystal composition comparison of the standard Bt kurstaki and Bt israeliensis with the Bt strain LRC3 (FIG. 1). Purified crystals for the three strains were run on NuPAGE™ Mops-Novex 10% Bis-Tris gel (Invitrogen). FIG. 1 shows the protein banding patterns for crystals isolated from Bt strain LRC3 (lane 2), Bt kurstaki (lane 3) and Bt israeliensis (lane 4), with molecular weight standards (kDa) in lane 1 (myosin 191, phosphorylase 97, bovine serum albumin 64, glutamic dehydrogenase 51, alcohol dehydrogenase 39, carbonic anhydrase 28, myoglobin red 19, lysozyme 14). Bt kurstaki has about a 191, 120 kDa bands representing Cry1Aa, Cry1Ab and Cry1Ac, and about a 64 kDa band representing Cry2A. Bt israeliensis has bands of about 100 kDa representing Cry4A; a 97 kDa protein representing Cry4B; a 64 kDa protein representing Cry 10 and Cry11; and a 30 or 27 kDa band representing Cyt1A toxin. There is also about a 19 kDa protein band that has not been observed in samples run on standard SDS-gel systems. Bt strain LRC3 has about a 191 kDa band, 120 kDa band, 80 kDa band, 70 kDa band, 55 kDa band, 48 kDa band, 40 kDa band, 38 kDa band, 28 kDa band and 25 kDa band.

### EXAMPLE 3

### DNA Fingerprinting for Comparison of Bt Strain LRC3 to Bt kurstaki and Bt israeliensis

a) PCR Mixtures for REP, BOX and ERIC Primers

Three different typing methods were used to generate fingerprinting patterns, namely REP, ERIC and RAPD with each method having its own specialized primer sequences (Table 3). Standard REP-PCR mixtures were made for all primers (Urzi et al., 2001) except the Bc-REP-PCR mixtures (Reyes-Ramirez and Ibarra, 2005).

TABLE 3

Sequence of primers used for the
fingerprinting amplifications

| | |
|---|---|
| Size | |
| (number | Melting |
| of | Temperature |

| | | | |
|---|---|---|---|
| Primer | Sequence | bases) | (° C.) |

| | | | |
|---|---|---|---|
| BOX-A1R | CTACGGCAAGGCGACGCTGACG | 22 | 64.8 |
| REP-1R | IIIICGICGICATCIGGC | 18 | 68.2 |
| REP-2I | ICGICTTATCIGGCCTAC | 18 | 57.5 |
| Bc-REP-1 | ATTAAAGTTTCACTTTAT | 18 | 37.7 |
| Bc-REP-2 | TTTAATCAGTGGGG | 14 | 39.8 |
| ERIC-1R | ATGTAAGCTCCTGGGGATTCAC | 22 | 56.7 |
| ERIC-2 | AAGTAAGTGACTGGGGTGAGCG | 22 | 59 |
| 0910-08 | CCGGCGGCG | 9 | 49.1 |
| 0940-12 | ACGCGCCCT | 9 | 42.8 |
| 0955-03 | CCGAGTCCA | 9 | 30.8 |

b) PCR Mixtures for RAPD Primers

RAPD can be a difficult procedure, requiring testing of various protocols for each primer used to obtain optimal amplified band production. The method of Nilsson et al., 1998 was used for the 0955-03 and 0940-12 primers, while the method of Brousseau et al., 1993 was used for the primer 0910-08.

c) PCR Amplification Conditions for REP, BOX and ERIC Primers

All fingerprinting amplification conditions were identical with any variations occurring only in the annealing reaction due to the variable size of the primers. The following PCR program was used: initial denaturation at 95° C. for 5 min, followed by 35 cycles of denaturation (95° C. for 1 min), primer annealing for 2 min at the appropriate temperature (see Table 4), and polymerization (72° C. for 2 min). The final amplification was at 72° C. for 10 min.

d) Agarose Gel Electrophoresis

Prior to electrophoresis, Vistra Green™ (Amersham Life Science, RPN 5786) was added directly to each PCR-generated sample at a final concentration of 1× (concentrate is 10,000×). Each sample was run on a 1.2% agarose gel (Multicell 400-700115) for 1 hour at 100V and then at 175V for an additional 2 hours. The gel was visualized using the Kodak™ Gel Logic 200 imaging system using the 535 nm WB50 SYBR Green filter.

TABLE 4
Annealing temperature used for fingerprinting primers
and primer pairs

| | Annealing |
|---|---|
| Primer | Temperature |
| BOX-A1R | 45° C. |
| ERIC-2 | 45° C. |
| ERIC-1R | 45° C. |
| ERIC-2 & BOX-A1R | 45° C. |
| ERIC-2 & ERIC-1R | 45° C. |
| Bc-REP-1 | 42° C. |
| Bc-REP-2 | 30° C. |
| Bc-REP-1 & Bc-REP-2 | 42° C. |
| REP-1R | 45° C. |
| REP-2I | 45° C. |
| REP-2I & REP-1R | 45° C. |
| 0955-03 | 40° C. |
| 0910-08 | 40° C. |
| 0940-12 | 40° C. |

e) Fluorescent DNA Labelling

The genomic DNA was labeled with the Bioprime™ DNA labeling System (Invitrogen) which uses random primers to linearly amplify and label genomic DNA in one reaction. The biotin-dNTP mix provided with the kit was replaced by a homemade dNTP mix and Cy5-dCTP (Perkin-Elmer) to fluorescently label the DNA in one step. The reaction was otherwise performed according to the manufacturer's protocol and incubated for 3.5 hours at 37° C. The reactions were purified using a PureLink™ PCR Purification Kit (Invitrogen). The DNA yield and purity, as well as the incorporation of the dye were assessed by measuring the optical density at 260 nm (DNA) and 650 nm (Cy5 dye), and by calculating the 260 nm/280 nm ratio using the NanoDrop ND-1000 spectrophotometer (NanoDrop Technologies). Only DNA showing a cyanine dye incorporation between 3.9-4.7% was used for hybridization (which is between 0.7-1.0 pmol/ul dye incorporation, see http://www.pangloss.com/seidel/Protocols/percent_inc. html).

f) cryArray Hybridization and Analyses

For hybridization on the cryArray version 11.0 chip, a pre-hybridization step was first performed with DIG™ Easy Hyb Buffer (Roche) in the presence of 5% BSA (Gibco) for 1 hour at 47° C. in a water bath. The slide was then immersed into 0.1×SSC pre-heated to 37° C. to remove the coverslip and dried using an air duster. All of the labeled DNA generated during the Bioprime labeling reaction (1.3 to 2.1 μg depending on the sample) was used for the hybridization. The DNA was dried in a SpeedVac and resuspended in DIG buffer. The DNA was denatured at 95° C. for 5 minutes and put on ice for 5 minutes prior to the 4 hour hybridization at 47° C. (Letowski et al., 2005). The coverslips were removed using 0.1×SSC/0.2% SDS pre-heated to 37° C. and the slide was washed three times with 0.1×SSC/0.2% SDS for 5 minutes at 37° C. A final wash with SSC 0.1× was done for 5 minutes at 37° C. before drying the slide with an air duster.

g) Results

FIG. 2 shows the DNA fingerprint of Bt strain LRC3 (lane 4) compared to Bt kurstaki (lane 1) and Bt israeliensis (lane 3) using REP-1R and REP-21 primers individually (section A and B) and in combination (section C). Eight μl of each reaction was loaded per lane. Lane M is the DNA size ladder in kilobases (kb), while lane C is the control (-DNA template). The pattern produced by the REP-1R primer suggested that Bt strain LRC3 and Bt israeliensis were very similar; however, Bt israeliensis DNA produced a strong band at 1.5 kb that was absent in Bt strain LRC3.

A similar scenario was observed with the REP-2I primer where all strains shared many common bands, but Bt strain LRC3 could be differentiated from Bt kurstaki by a strong band at 0.2 kb and from Bt israeliensis by lacking two bands between 1.3 and 1.6 kb. A combination of these two primers provided a weak single band for Bt israeliensis at ˜0.3 kb which allowed discrimination from the nearly identical Bt strain LRC3.

FIG. 3 shows the DNA fingerprint of Bt strain LRC3 (lane 4) compared to Bt kurstaki (lane 1) and Bt israeliensis (lane 2) using Bc-REP-1 and Bc-REP-2 primers, individually (sections A and B, respectively) and in combination (section C). Eight μl of each reaction was loaded per lane. Lane M is the DNA size ladder (in kb), and lane C is the control (-DNA template). Different patterns were observed using the Bacillus-based REP primers, Bc-REP-1 and Bc-REP-2. The Bc-REP-1 primer provided excellent discrimination of Bt strain LRC3 from Bt kurstaki by producing no common bands. Bt strain LRC3 and Bt israeliensis could be distinguished by having unique bands, 2.7 kb and 2.2 kb, respectively. Bc-REP-2 was an even better discriminator of the Bt strains. At this point, numerous similarities between the mosquitocidal Bt israeliensis and Bt strain LRC3 were observed; however, Bc-REP-2 clearly produced two strong bands for Bt strain LRC3 not seen in Bt israeliensis, which produced one strong unique band at ˜0.8 kb. Interestingly the strong bands seen with Bc-REP-2 for either Bt israeliensis or Bt strain LRC3 disappear when the two Bc-REP primers are mixed together making the two strains appear similar except for one strong and two weak identifying bands for Bt israeliensis.

FIG. 4 shows the DNA fingerprint of Bt strain LRC3 (lane 4) compared to Bt kurstaki (lane 1) and Bt israeliensis (lane 2) using Enterobacteriaceae-based REP primers ERIC-1 and ERIC-2, individually (sections A and B, respectively) and in combination (section C). Although more bands are produced among the Bt strains, many common bands are shared; however, unique bands discriminating Bt strain LRC3 from the other strains can be observed for the ERIC primers. When the two primers were combined, the banding pattern was more complex with many weak bands suggesting that this mixture does not discriminate as well as the individual primers.

FIG. 5 shows the DNA fingerprint of Bt strain LRC3 (lane 4) compared to Bt kurstaki (lane 1) and Bt israeliensis (lane 2) using ERIC amplification techniques with BOX-A1R primer individually (section A) and in combination with ERIC-2 primer (section B). The multiplicity of amplified bands seen with the ERIC primers was even higher when the BOX-A1R primer was used alone. This primer pattern resulted in Bt strain LRC3 having a unique 1.6 kb band. In addition, Bt strain LRC3 could be further distinguished from Bt israeliensis by having a 2.6 kb band and lacking a 1.9 kb band. Interestingly, using the BOX-A1R primer in conjunction with the ERIC-2 primer, the majority of the numerous bands above 1 kb seen with the BOX-A1R primer alone disappeared and new bands, not seen with either primer alone, appeared below 0.5 kb. These lower bands provide easy discrimination among the three isolates.

FIG. 6 shows the DNA fingerprint of Bt strain LRC3 (lane 4) compared to Bt kurstaki (lane 1) and Bt israeliensis (lane 2) using RAPD amplification techniques with 0955-03, 1940-12 and 1910-08 primers (sections A, B and C, respectively). Lane M is the DNA size ladder (in kb) and lane C is the control (-DNA template). Eight μl of each reaction was loaded per lane. The 0955-03 and 1940-12 produced excellent discriminatory patterns with no strong bands common among the strains. The 1910-08 primer produced a number of common bands among the strains; however, differences between Bt strain LRC3 and the other strains could be found on a case-by-case basis.

### EXAMPLE 4

### Cry Gene Content of Bt Strain LRC3, Bt kurstaki and Bt israeliensis

Prior to hybridization on a cryArray version 11.0 chip, a pre-hybridization step was first performed with DIG™ Easy Hyb Buffer (Roche) in the presence of 5% bovine serum albumin (Gibco) for 1 hr at 47° C. in a water bath. The slide was then immersed into 0.1×SSC pre-heated to remove the coverslip and dried using an airduster. All labelled DNA generated during the Bioprime™ labelling reaction (1.3 to 2.1 ug depending on the sample) was used for the hybridization. The DNA was dried in a Savant SpeedVac™ and resuspended in DIG buffer. The DNA was denatured at 95° C. for 5 min. and put on ice for 5 min prior to the 4 hr hybridization at 47° C. (Letowski et al., 2005). The coverslips were removed using 0.1×SSC/0.2% SDS preheated to 37° C. and the slide was washed three times with 0.1×SSC/0.2% SDS for 5 min at 37° C. A final wash with SSC 0.1× was done for 5 min at 37° C. before drying the slide with an air duster.

The cry gene content of Bt strain LRC3 was assessed using a cryArray chip, version 11.0 which concentrates on tertiary ranked Cry1 genes but also includes general primary ranked gene probes for cry2, cry3, cry4, cry9 and cry11. FIG. 7 shows the printing key for the cryArray results referred to as the cry microarray assay. All oligonucleotide probes were printed in triplicate with the tertiary cry1-specific probes printed in the top three quarters of the chip, the more general secondary ranked cry1 primers in the lower right quadrant of the chip and the other general primary ranked gene probes in the lower left quadrant. Purified genomic DNA from the four fingerprinted strains were labeled with a cyanine-5 dye and re-purified. Approximately 1.3-2.1 μg of each DNA sample was hybridized to the cryArray.

The first strain hybridized to the cryArray was the well known commercial strain Bt kurstaki (FIG. 8). A number of positive fluorescent spots can be observed. As constrained by the redundancy design of the array, all probes designed for a particular gene must be positive for that gene to be considered as present in the strain. This chip clearly identified two primary gene classes, cry1 and cry2. At the secondary level, the cry1 genes were divided among the cry1A and cry1I genes. Since this chip concentrated mainly on the large cry1 gene family, only one secondary cry2 gene probe was printed; thus, only a cry2A gene could be identified. At the tertiary level, the cry1 and cry2 signals were identified as the three known secondary classes of genes, cry1A genes (cry1Aa, b and c), the cry1Ia gene and the cry2Aa gene, all of which are known to be in this strain.

In accordance with the redundancy approach, genes which show only a subset of the gene specific oligos being positive or fluorescent are not considered present. Since Bt strain LRC3 is dipteran-active and showed the most similarity at the genomic level to Bt israeliensis (as shown by the fingerprinting data), hybridization with labeled Bt israeliensis was carried out (FIG. 9). The primary ranked cry4 and cry11 gene probes were positive.

When the labeled genomic DNA from Bt strain LRC3 was hybridized to the cryArray, numerous spots were positive corresponding to cry1 and cry2 genes (FIG. 10). As with Bt kurstaki and Bt israeliensis, a second hybridization was carried out to confirm the results of the first. The results are 100% consistent with the first hybridization (data not shown). Designing general primary and secondary ranked probes allows the investigator to detect new gene variants. For example, if probes for the secondary ranked cry1B are positive but none of the tertiary ranked cry1B probes (i.e., cry1Ba, cry1Bb etc.) are positive, a new variant of the gene exists. As the summary of the Bt strain LRC3 results show in Table 5 and 6, among the seven different primary classes examined, two were positive (cry1 and cry2). Within these two primary ranks, at least seven different genes at the tertiary level are present in this strain (Table 6). Furthermore, among these seven genes, a new variant of cry1A has been noted as deduced by the lack of positive tertiary cry1A ranked probes when the general secondary cry1A probes are clearly positive. At the cry2 level, the tertiary cry2Aa probe was negative while the primary probes were positive. Since all the secondary cry2 gene classes are not represented on the chip, it is inconclusive whether there is a new cry2 gene. The only conclusion that can be made is that it is not a cry2Aa gene (Table 6).

TABLE 5
Positive primary ranks or subclasses

| Primary ranks tested | Primary ranks positive on cryArray^{a} |
|---|---|
| cry1 | + |
| cry2 | + |
| cry3 | − |
| cry4 | − |
| cry9 | − |
| cry11 | − |

^{a}+ = present; − = absent

TABLE 6
Tertiary rank summary of positive genes

| | Secondary | | |
|---|---|---|---|
| Primary cry rank | cry rank | Tertiary cry rank | Host specificity^{c} |
| cry1 | cry1A | ?^{a} | Lepidoptera |
| cry1 | cry1B | cry1Bb | ? |
| cry1 | cry1F | cry1Fb | ? |
| cry1 | cry1H | cry1Hb | ? |
| cry1 | cry1I | cry1Ic | ? |
| cry1 | cry1K | cry1Ka | ? |
| cry2 | cry2 | ?^{b} | Diptera |

^{a}Although a cry1A secondary class gene was detected, it does not fall into any of the known cry1A classes and must be considered a novel gene.
^{b}Although a cry2 primary class gene was detected, probes specific to cry2 genes other than cry2Aa were not on the chip and thus we can only conclude that it is not a cry2Aa gene.
^{c}Host specificity was checked at either the Bacillus thuringiensis crystal toxin gene nomenclature site (http://www.lifesci.sussex.ac.uk/home/Neil_Crickmore/Bt/) (redirected to NCBI) or the CFS toxicity database website (http://www.glfc.cfs.nrcan.gc.ca/science/research/netintro99_e.html). The question mark means the specificity is not public knowledge or is unknown.

### EXAMPLE 5

### Insecticidal Activity of Bt Strain LRC3 Against Adult Insects of the Order Diptera

The insecticidal activity of Bt strain LRC3 was compared to that of Bt israeliensis (BGSC-4Q5) by conducting feeding bioassays on adult house flies and stable flies. Adult horn flies were not tested as they cannot be reared off animals, while adult face flies were also not included since the crystal proteins needed to have been formulated for a dry diet. The Bt strain LRC3 and Bt israeliensis were used in their entirety (i.e., including spores/crystals/bacterial products). Thirty newly-emerged adult house flies or stable flies were added to a bioassay chamber and provided with Bt strain LRC3 or Bt israeliensis at a dose of about 10⁷ org/ml in the normal diet (i.e., evaporated milk for the house flies and blood for the stable flies). For three days, all flies were provided with fresh food/bacteria and any dead flies were removed. All flies were assessed after three days exposure to the treatment (Table 7). Bt strain LRC3 was found to be active against both adult house and stable flies, whereas Bt israeliensis was ineffective.

TABLE 7
Insecticidal activity of the Bt strain LRC3
against adult insects of the Order Diptera

| Dipteran | Bt LRC3 strain | Bt israelensis |
|---|---|---|
| Species | Percent Mortality | Percent Mortality |
| House fly | 57 | 0 |
| Stable fly | 100 | 0 |

### EXAMPLE 6

### Purification of Crystals from Bt Strain LRC3 and Bt israeliensis (BGSC-4Q5)

The crystals from Bt strain LRC3 and Bt israeliensis were isolated and purified. Each strain was cultured in 335 ml of nutrient broth (Example 1, Table 2). Three flasks were grown for each organism from the working stocks and were shaken at 200 rpm at 28° C. for 7 days. The cultures were harvested by centrifugation at 7000 rpm for 20 min in 250 ml centrifuge bottles. The pellets from the 3 flasks per strain were then pooled into one 250 ml centrifuge bottle and 100 ml of sterile fly physiological saline was added. To each bottle, 0.4 g of lysozyme was added and the bottles were placed on a shaker at 200 rpm for 2 days. The bottle was centrifuged at 10,000 rpm for 20 min and the supernatant was discarded. 100 ml of a saline wash solution (0.1M Tris, 1M NaCl, 10 mM EDTA, pH 7.0) and 1% Triton TMX 100 were added and the bottle was placed on a stir plate for 2 days. The following steps of centrifugation, resuspension or washing of the pellet in specific buffer, and stirring were then conducted:
a) Centrifugation, resuspension of the pellet in 200 ml of saline wash solution, and stirring for 1 day;
b) Centrifugation, resuspension of the pellet in 200 ml of 50% saline wash solution, and stirring for 1 day;
c) Centrifugation, resuspension of the pellet in 100 ml of 50% saline wash solution, addition of 0.2 g lysozyme, and stirring for 2 days;
d) Centrifugation, washing of the pellet twice with 200 ml of 50% saline wash solution;
e) Centrifugation, washing of the pellet twice with 200 ml of 25% saline wash solution; and
f) Centrifugation, washing of the pellet twice with 200 ml of sterile distilled water.
   The purified crystal pellet was finally resuspended in 25-50 ml of sterile distilled water. For long term storage of the crystals, the addition of 0.02% sodium azide is recommended. The final preparation was checked for purity by running samples on 10% SDS-PAGE gels, and the protein concentration of the preparation was determined using the Bio-Rad™ Protein Assay (Bio-Rad Laboratories, 1000 Alfred Nobel Drive, Hercules, Calif., U.S.A. 94547).

### EXAMPLE 7

### Insecticidal Activity of Purified Crystals of Bt Strain LRC3 Against Adult Insects of the Order Diptera

The effect of the purified crystals of Bt strain LRC3 against adult house flies and stable flies was tested. Bt israeliensis crystals were not tested as the bacterium was not active. Thirty newly-emerged adult house flies or stable flies were added to a bioassay chamber and provided with purified crystals of the Bt strain LRC3 at a dose of about 389 ng crystal protein/ml in the normal diet (i.e., evaporated milk for the house flies and blood for the stable flies). For three days, all flies were provided with fresh food/purified crystals and any dead flies were removed. All flies were assessed after three days exposure to the treatment (Table 8).

TABLE 8
Insecticidal activity of purified crystals of Bt strain LRC3
against adult insects of the Order Diptera

| Dipteran species | Percent Mortality | LD₅₀ (ng) |
|---|---|---|
| House fly | 51 | 389 |
| Stable fly | 95 | 34 |

LD₅₀ values (i.e., the amount of purified crystals which induce mortality in 50% of the group of test flies) were obtained to measure the short-term poisoning potential or acute toxicity of the purified crystals. Based on the LD₅₀ values, adult stable flies appear to be 10× more sensitive to purified crystals of Bt strain LRC3 than are adult house flies.

### EXAMPLE 8

### Insecticidal Activity of Bt Strain LRC3 Against Immature Insects of the Order Diptera

Bt strain LRC3 strain was cultured as described in Example 1, and centrifuged and resuspended in sterile water to provide a preparation with about 108 spores/ml. This bacterial preparation was then mixed with the rearing diet of each fly species at a dose of 2.5×10⁷ spores/g diet. Control treatments consisted of sterile water.

i. Rearing Bioassays

For face fly, 35 eggs were placed on filter paper and then placed upside down on treated cattle manure. For fruit fly, males and females were obtained from a laboratory colony and four pairs were placed in a standard fruit fly rearing vial. Pupation was assessed from 1 to 2 weeks after the start of the experiment. For horn fly, 25 eggs were placed on filter paper and then placed upside down on treated cattle manure. House fly eggs were collected from a laboratory colony and washed, and the hatching first-instar larvae were collected. About 30 first-instar larvae were placed on filter paper and then placed upside down on treated artificial fly rearing diet (i.e., wheat bran, brewer's grain, alfalfa, brewer's yeast, and water). Stable fly eggs were collected from a lab colony, washed and placed on filter paper on a nutrient agar plate. The eggs were incubated overnight at 25° C., 60% relative humidity and a 16:8 photoperiod. Newly emerged first-instar larvae were collected after about 24 hrs. About 30 first-instar larvae were placed on filter paper and then placed upside down on treated artificial fly rearing diet (i.e., wheat bran, sawdust, brewer's grain, alfalfa, brewer's yeast, water).

ii. Ring Bioassay

About 30 first-instar house fly or stable fly larvae were reared and then placed on filter paper on an egg-yolk medium plate made by combining 1.0 g/L peptone, 2.0 g/L NaCl, 0.26 g/L KH₂PO₄, 2.6 g/L Na₂HPO₄, 15 g/L agar and 4 egg yolks/L, and then pouring the final mixture into standard Petri dishes and cooling to room temperature. Food was supplied for the fly larvae by seeding the egg yolk plates with equal amounts of Empedobacter and Flavobacter. The plates were placed in a tub and covered with a piece of paper towel. The plates were then incubated at 28° C., 60% relative humidity and a 16:8 photoperiod for about one week for the house flies and two weeks for the stable flies. The plates were then checked for pupae (Table 9). In Table 5, “ND” designates that the experiments were not conducted for reasons associated with different fly behaviour and rearing capabilities.

TABLE 9
Insecticidal activity of Bt strain LRC3
against immature insects of the Order Diptera

| | Rearing Diet Bioassay | | Ring Bioassay | |
|---|---|---|---|---|
| | Bt LRC3 strain | Bt israeliensis | LRC3 | Bt israeliensis |
| Dipteran | Percent | Percent | Percent | Percent |
| Species | Mortality | Mortality | Mortality | Mortality |
| Face fly | 100 | ND | ND | ND |
| Fruit fly | 100 | ND | ND | ND |
| Horn fly | 100 | 50 | ND | ND |
| House fly | 94 | 0 | 67 | 56 |
| Stable fly | 98 | 0 | 74 | 85 |

The results demonstrate that both Bt strain LRC3 and Bt israeliensis display similar activity in simple diets. However, Bt strain LRC3 is more effective than Bt israeliensis against non-aquatic immature flies in complex rearing environments, suggesting that Bt strain LRC3 is better able to survive in a complex environment than Bt israeliensis. Further, the results demonstrate that with the correct assay system, it was found that specific Bt strains have activity against higher flies. As inappropriate assay systems have been used in the prior art, the activity of these particular Bt strains against higher flies had previously remained undetected.

### EXAMPLE 9

### Insecticidal Activity of Purified Crystals of Bt Strain LRC3 Against Immature Insects of the Order Diptera

The effect of the purified crystals of Bt strain LRC3 was tested against immature house flies and stable flies. The test was performed as described in Example 6 (see “Ring Bioassay”). Results are shown in Table 10.

TABLE 10
Insecticidal activity of purified crystals of Bt strain LRC3
against immature insects of the Order Diptera

| Dipteran | LRC3 | Bt strain LRC3 | Bt israeliensis |
|---|---|---|---|
| species | Percent Mortality | LD₅₀ (ng) | Percent Mortality |
| House fly | 80 | 0.44 | 22 |
| Stable fly | 100 | 0.1 | 84 |

### REFERENCES

Ausubel, F. M., Bent, R., Kingston, R. E., Moore, D. J., Smith, J. A., Silverman, J. G. and Struhl K. (2000) Current Protocols in Molecular Biology. John Wiley & Sons, New York.
Brousseau, R., Saint-Onge, A., Prefontaine, G., Masson, L. and Cabana, J. (1993) Arbitrary primer-polymerase chain reaction: a powerful method to identify Bacillus thuringiensis serovars and strains. Appl. Env. Microbiol. 59(1):114-119.
Hofte, H., VanRie, J., Jansens, S., Van Houtven, A., Vanderbruggen, H. and Vaeck, M. (1988) Monoclonal antibody analysis and insecticidal spectrum of three types of Lepidopteran-specific insecticidal crystal proteins of Bacillus thuringiensis. Appl. Env. Micro. 54(9):2010-2017.
Keller, B. and G. A. Langenbruch (1993) Control of Coleopteran Pests by Bacillus thuringiensis. In Bacillus thuringiensis, an Environmental Biopesticide: Theory and Practice. P. F. Entwistle, J. S. Cory, M. J. Bailey and S. Higgs (eds.), Jon Wiley & Sons Ltd., London, pp. 171-191.
Letowski, J., Bravo, A., Brousseau, R., and Masson, L. (2005) Rapid assessment of cry1 gene content of Bacillus thuringiensis strains using DNA microarrays. Appl. Env. Microbiol. In press.
Nilsson, J., Svensson, B., Ekelund, K. and Christiansson, A. (1998) A RAPD-PCR method for large-scale typing of Bacillus cereus. Lett. Appl. Microbiol. 27(3):168-172.
Reyes-Ramirez, A. and Ibarra, J. E. (2005) Fingerprinting of Bacillus thuringiensis type strains and isolates by using Bacillus cereus group-specific repetitive extragenic palindromic sequence-based PCR analysis. Appl. Env. Microbiol. 71(3):1346-1355.
Sambrook, J., Fritsch, E. F. and Maniatis, T. (1989) Molecular Cloning, A Laboratory Manual. Cold Spring Harbour Laboratory Press.
Sanchis, V., Lereclus, D., Menou, G., Chaufaux, J. and Lecadet, M. M. (1988) Multiplicity of beta-endotoxin genes with different insecticidal specificities in Bacillus thuringiensis aizawai 7.29. Mol. Microbiol. 2(3):393-404.
Urzi, C., Brusetti, L., Salamone, P., Sorlini, C., Stackebrandt, E., and Daffonchio, D. (2001) Biodiversity of Geodermatophilaceae isolated from altered stones and monuments in the Mediterranean basin. Environ. Microbiol. 3:471-479.
Versalovic, J., Koeuth, T., McCabe, E. R. B. and Lupski, J. R. (1991a) Use of the polymerase chain reaction for physical mapping of Escherichia coli genes. J. Bacteriol. 173(17):5253-5255.

Versalovic, J., Koeuth, T. and Lupski, J. R. (1991b) Distribution of repetitive DNA sequences in eubacteria and application to fingerprinting of bacterial genomes. Nuc. Acid Res. 19(24):6823-6831.
Visser, B., Munsterman, E., Stoker, A. and Dirkse, W. G. (1990) A novel Bacillus thuringiensis gene encoding a Spodoptera exigua-specific crystal protein. J. Bacteriol. 172(12):6783-6788.

### PATENT DOCUMENTS

Baum, J. A., Chu, C., Donovan, W. P., Gilmer, A. J., and Rupar, M. J. Lepidopteran-active Bacillus thuringiensis d-endotoxin compositions and methods of use. U.S. Pat. No. 6,593,293; issued Jul. 15, 2003.
Brown, K. L., Kit L. and Whiteley, H. R. Crystal proteins of Bacillus thuringiensis, genes encoding them, and host expressing them. U.S. Pat. No. 5,308,760; issued May 3, 1994.
Donovan, W. P. and Baum, J. A. Constructed Bacillus thuringiensis strains producing mosquitocidal crystal proteins. U.S. Pat. No. 6,482,636; issued Nov. 19, 2002.
Edwards, D. L.; Payne, J. and Soares, G. G. Novel isolates of bacillus thuringiensis having activity against nematodes. U.S. Pat. No. 4,948,734; issued Aug. 14, 1990.
Herrnstadt, C. and Soares, G. G. Control of cotton boll weevil, alfalfa weevil, and corn rootworm via contact with a strain of Bacillus thuringiensis. U.S. Pat. No. 4,797,276; issued Jan. 10, 1989.
Herrnstadt, C. and Wilcox, E. Cloning and expression of Bacillus thuringiensis toxin gene toxic to beetles of the order Coleoptera. U.S. Pat. No. 4,853,331; issued Aug. 1, 1989.
Hickle, L. A. and Payne, J. Materials and methods for the control of calliphoridae pests. U.S. Pat. No. 5,888,503; issued Mar. 30, 1999.
Lambert, B., Jansens, S., Van Audenhove, K., Peferoen, M., Van Rie, J. and Van Aarssen, R. Bacillus thuringiensis strains and their insecticidal proteins. U.S. Pat. No. 6,448,226; issued Sep. 10, 2002.
Payne, J. and Soares, G. G. Novel coleopteran-active bacillus thuringiensis isolate. U.S. Pat. No. 4,999,192; issued Mar. 12, 1991.
Payne, J. M. Isolates of Bacillus thuringiensis that are active against nematodes. U.S. Pat. No. 5,151,363; issued Sep. 29, 1992.
Schnepf, H. E., Wicker, C., Narva, K. E., Walz, M. and Stockhoff, B. A. Toxins active against pests. U.S. Pat. No. 6,570,005; issued May 27, 2003.
Soares, G. G.; Everich, R. C. and Payne, J. Novel isolates of bacilus thuringiensis having activity against the alfalfa weevil, hypera brunneipennis. U.S. Pat. No. 4,849,217; issued Jul. 18, 1989.

All publications mentioned in this specification are indicative of the level of skill in the art to which this invention pertains. All publications are herein incorporated by reference to the same extent as if each individual publication was specifically and individually indicated to be incorporated by reference.

Although the foregoing invention has been described in some detail by way of illustration and example, for purposes of clarity and understanding it will be understood that certain changes and modifications may be made without departing from the scope or spirit of the invention as defined by the following claims.

What is claimed is:

1. A method for controlling an insect of the Order Diptera comprising the step of:
a) providing a Bacillus thuringiensis strain or a variant thereof, or a spore or a crystal of the Bacillus thuringiensis strain or a variant thereof, the Bacillus thuringiensis strain containing a plasmid carrying one or more endotoxin genes for encoding one or more delta-endotoxins selected from the group consisting of Cry1A, Cry1B, Cry1F, Cry1H, Cry1I, Cry1K, Cry2 and a variant thereof; and one of the following steps selected from:
b) contacting the insect with the Bacillus thuringiensis strain or the variant thereof, or the spore or the crystal of the Bacillus thuringiensis strain or the variant thereof;
c) applying the Bacillus thuringiensis strain or the variant thereof, or the spore or the crystal of Bacillus thuringiensis strain or the variant thereof to an infested area; or
d) administering the Bacillus thuringiensis strain or the variant thereof, or the spore or the crystal of Bacillus thuringiensis strain or the variant thereof to an animal.

2. The method of claim 1, wherein the insect is an immature or adult insect selected from the group consisting of Nematocera, Bracycera and Cyclorrhapha.

3. The method of claim 2, wherein the insect is selected from the group consisting of black fly, crane fly, gnat, midge, mosquito, sand fly, bee fly, deer fly, horse fly, robber fly, bottle fly, blow fly, cattle grub, deer ked, house fly, face fly, fruit fly, horn fly, horse louse fly, human bot fly, rodent fly, rabbit bot fly, sheep ked, sheep nasal bot, stable fly, stomach bot and Tsetse fly.

4. The method of claim 3, wherein the one or more endotoxin genes are selected from the group consisting of cry1A, cry1Bb, cry1Fb, cry1Hb, cry1Ic, cry1Ka, cry2 and a variant thereof.

5. The method of claim 4, wherein the delta-endotoxins are all of Cry1A, Cry1B, Cry1F, Cry1H, Cry1I, Cry1K and Cry2.

6. The method of claim 5, wherein the endotoxin genes are all of cry1A, cry1Bb, cry1Fb, cry1Hb, cry1Ic, cry1Ka and cry2.

7. The method of claim 6, wherein the strain is Bacillus thuringiensis strain LRC3 deposited as ATCC PTA-6248.

8. The method of claim 1, wherein the animal is selected from the group consisting of dairy cow, beef cow, pig, goat, sheep, horse, deer, buffalo, elk, chicken, turkey, cat, dog, and horse.

9. The method of claim 7, wherein the Bacillus thuringiensis strain or the variant thereof, or the spore or the crystal of Bacillus thuringiensis strain or the variant thereof is provided in the form of a solid, liquid, feed additive or feed composition.

10. The method of claim 9, wherein the Bacillus thuringiensis strain or the variant thereof, or the spore or the crystal of Bacillus thuringiensis strain or the variant thereof is provided in combination with adhesion agents, binders, botanical materials, carriers, detergents, diluents, dispersants, emulsifiers, excipients, extenders, fillers, inorganic minerals, insecticidal carriers, polymers, rheological agents, spreader sticker adjuvants, stabilizing agents, surfactants, pesticidal additives, wetting agents, Bt cells, crystals, crystal proteins, protoxins, toxins, biocides, fertilizers, fungicides, herbicides or combinations thereof.

11. The method of claim 10, comprising providing the Bacillus thuringiensis strain or the variant thereof, or the spore or the crystal of the Bacillus thuringiensis strain or variants thereof in the form of a solid at a concentration of 1-95% by weight or volume of the strain, spore, crystal or variant, more preferably 2-80% by weight or volume of the strain, spore, crystal or variant, and most preferably 30-60% by weight or volume of the strain, spore, crystal or variant.

12. The method of claim 10, comprising providing the Bacillus thuringiensis strain or the variant thereof, or the spore or the crystal of the Bacillus thuringiensis strain or variants thereof in the form of a liquid at a concentration of 1-60% by weight or volume of strain, spore, crystal or variant, more preferably 10-50% by weight or volume of strain, spore, crystal or variant, and most preferably 20-40% by weight or volume of strain, spore, crystal or variant.

13. The method of claim 10, comprising providing the Bacillus thuringiensis strain or the variant thereof, or the spore or the crystal of the Bacillus thuringiensis strain or variant thereof at a concentration of 10²-10⁴ spores/ml, more preferably 200-800 spores/ml, and most preferably 300-700 spores/ml.

14. The method of claim 10, comprising providing the Bacillus thuringiensis strain or the variant thereof, or the spore or the crystal of the Bacillus thuringiensis strain or variant thereof at a concentration of at least 50 g per hectare and more preferably at least 1 kg per hectare.

15. A composition for controlling an insect of the Order Diptera comprising a Bacillus thuringiensis strain or a variant thereof, or a spore or a crystal of Bacillus thuringiensis strain or a variant thereof, the Bacillus thuringiensis strain containing a plasmid carrying one or more endotoxin genes for encoding one or more delta-endotoxins selected from the group consisting of Cry1A, Cry1B, Cry1F, Cry1H, Cry1I, Cry1K, Cry2 and a variant thereof.

16. The composition of claim 15, wherein the insect is an immature or adult insect selected from the group consisting of Nematocera, Bracycera and Cyclorrhapha.

17. The composition of claim 16, wherein the insect is selected from the group consisting of black fly, crane fly, gnat, midge, mosquito, sand fly, bee fly, deer fly, horse fly, robber fly, bottle fly, blow fly, cattle grub, deer ked, house fly, face fly, fruit fly, horn fly, horse louse fly, human bot fly, rodent fly, rabbit bot fly, sheep ked, sheep nasal bot, stable fly, stomach bot and Tsetse fly.

18. The composition of claim 17, wherein the one or more endotoxin genes are selected from the group consisting of cry1A, cry1Bb, cry1Fb, cry1Hb, cry1Ic, cry1Ka, cry2 and a variant thereof.

19. The composition of claim 18, wherein the delta-endotoxins are all of Cry1A, Cry1B, Cry1F, Cry1H, Cry1I, Cry1K and Cry2.

20. The composition of claim 19, wherein the endotoxin genes are all of cry1A, cry1Bb, cry1Fb, cry1Hb, cry1Ic, cry1Ka and cry2.

21. The composition of claim 20, wherein the strain is Bacillus thuringiensis strain LRC3 deposited as ATCC PTA-6248.

22. The composition of claim 21, wherein the Bacillus thuringiensis strain or the variant thereof, or the spore or the crystal of the Bacillus thuringiensis strain or the variant thereof is in the form of a solid, liquid, feed additive or feed composition.

23. The composition of claim 22, further comprising adhesion agents, binders, botanical materials, carriers, detergents, diluents, dispersants, emulsifiers, excipients, extenders, fillers, inorganic minerals, insecticidal carriers, polymers, rheological agents, spreader sticker adjuvants, stabilizing agents, surfactants, pesticidal additives, wetting agents, Bt cells, crystals, crystal proteins, protoxins, toxins, biocides, fertilizers, fungicides, herbicides or combinations thereof.

24. The composition of claim 23, comprising the Bacillus thuringiensis strain or the variant thereof, or the spore or the crystal of the Bacillus thuringiensis strain or the variant thereof in the form of a solid at a concentration of 1-95% by weight or volume of the strain, variant, or crystal thereof, more preferably 2-80% by weight or volume of the strain, variant, or crystal thereof, and most preferably 30-60% by weight or volume of the strain, variant, or crystal thereof.

25. The composition of claim 23, comprising the Bacillus thuringiensis strain or the variant thereof, or the spore or the crystal of the Bacillus thuringiensis strain or the variant thereof in the form of a liquid at a concentration of 1-60% by weight or volume of the strain, variant, or crystal thereof, more preferably 10-50% by weight or volume of the strain, variant, or crystal thereof, and most preferably 20-40% by weight or volume of the strain, variant, or crystal thereof.

26. The composition of claim 23, comprising the Bacillus thuringiensis strain or the variant thereof, or the spore or the crystal of the Bacillus thuringiensis strain or the variant thereof at a concentration of 10²-10⁴ spores/ml, more preferably 200-800 spores/ml, and most preferably 300-700 spores/ml.

27. The composition of claim 23, comprising the Bacillus thuringiensis strain or the variant thereof, or the spore or the crystal of the Bacillus thuringiensis strain or the variant thereof at a concentration of at least 50 g per hectare, and more preferably at least 1 kg per hectare.

28. A crystal of a Bacillus thuringiensis strain or a variant thereof for use in controlling an insect of the Order Diptera, the crystal containing one or more delta-endotoxins selected from the group consisting of Cry1A, Cry1B, Cry1F, Cry1H, Cry1I, Cry1K, Cry2 and a variant thereof.

29. The crystal of claim 28, wherein the Bacillus thuringiensis strain contains a plasmid carrying one or more endotoxin genes for encoding one or more delta-endotoxins selected from the group consisting of Cry1A, Cry1B, Cry1F, Cry1H, Cry1I, Cry1K, Cry2 and a variant thereof.

30. The crystal of claim 29, wherein the one or more endotoxin genes are selected from the group consisting of cry1A, cry1Bb, cry1Fb, cry1Hb, cry1Ic, cry1Ka, cry2 and a variant thereof.

31. The crystal of claim 30, wherein the delta-endotoxins are all of Cry1A, Cry1B, Cry1F, Cry1H, Cry1I, Cry1K and Cry2.

32. The crystal of claim 31, wherein the endotoxin genes are all of cry1A, cry1Bb, cry1Fb, cry1Hb, cry1Ic, cry1Ka and cry2.

33. The crystal of claim 32, wherein the strain is Bacillus thuringiensis strain LRC3 deposited as ATCC PTA-6248.

34. An isolated nucleic acid of a Bacillus thuringiensis strain or a variant thereof, wherein the nucleic acid encodes a protein toxic to an insect of the Order Diptera, the protein being selected from the group consisting of Cry1A, Cry1B, Cry1F, Cry1H, Cry1I, Cry1K, Cry2 and a variant thereof.

35. The isolated nucleic acid of claim 34, wherein the nucleic acid comprises an endotoxin gene selected from the group consisting of cry1A, cry1Bb, cry1Fb, cry1Hb, cry1Ic, cry1Ka, cry2 and a variant thereof.

36. The isolated nucleic acid of claim 35, wherein the strain is Bacillus thuringiensis strain LRC3 deposited as ATCC PTA-6248.

37. A plasmid of claim 1 comprising one or more endotoxin genes selected from the group consisting of cry1A, cry1Bb, cry1Fb, cry1Hb, cry1Ic, cry1Ka, cry2 and a variant thereof.

38. The plasmid of claim 37, wherein the one or more endotoxin genes encode one or more delta-endotoxins selected from the group consisting of Cry1A, Cry1B, Cry1F, Cry1H, Cry1I, Cry1K, Cry2 and a variant thereof.

39. The plasmid of claim 38, wherein the delta-endotoxins are all of Cry1A, Cry1B, Cry1F, Cry1H, Cry1I, Cry1K and Cry2.

40. The plasmid of claim 39, wherein the endotoxin genes are all of cry1A, cry1Bb, cry1Fb, cry1Hb, cry1Ic, cry1Ka and cry2.

41. The plasmid of claim 40, wherein the strain is Bacillus thuringiensis strain LRC3 deposited as ATCC PTA-6248.

42. A vector comprising the nucleic acid of claim 34.

43. An isolated host cell comprising the nucleic acid of claim 34 or a plasmid having one or more endotoxin genes selected from the group consisting of cry1A, cry1Bb, cry1Fb, cry1Hb, cry1Ic, cry1Ka, cry2 and a variant thereof.

44. The host cell according to claim 43, wherein the cell is selected from the group consisting of Aspergillus niger, Aspergillus ficuum, Aspergillus awamori, Aspergillus oryzae, Bacillus subtilis or licheniformis, Clavibacterxyli, Escherichia coli, Kluyveromyces lactis, Mucor miehei, Pichia pastoris, Pseudomonas fluorescens, Saccharomyces cerevisiae, Trichoderma reesei, and a plant cell.

45. A Bacillus thuringiensis strain of claim 1, wherein the strain contains a plasmid carrying one or more endotoxin genes for encoding one or more delta-endotoxins selected from the group consisting of Cry1A, Cry1B, Cry1F, Cry1H, Cry1I, Cry1K, Cry2 and a variant thereof.

46. The strain of claim 45, wherein the one or more endotoxin genes are selected from the group consisting of cry1A, cry1Bb, cry1Fb, cry1Hb, cry1Ic, cry1Ka, cry2 and a variant thereof.

47. The strain of claim 46, wherein the delta-endotoxins are all of Cry1A, Cry1B, Cry1F, Cry1H, Cry1I, Cry1K and Cry2.

48. The strain of claim 47, wherein the endotoxin genes are all of cry1A, cry1Bb, cry1Fb, cry1Hb, cry1Ic, cry1Ka, and cry2.
